(19) United States
(12) Patent Application Publication
(10) Pub. No.: US 2022/0024992 A1
(43) Pub. Date: Jan. 27, 2022

(54) COMPOSITIONS AND METHODS COMPRISING LYSOCINS AS BIOENGINEERED ANTIMICROBIALS FOR USE IN TARGETING GRAM-NEGATIVE BACTERIA

(71) Applicants: The Rockefeller University, New York, NY (US); ContraFect Corporation, Yonkers, NY (US)

(72) Inventors: Vincent FISCHETTI, West Hempstead, NY (US); Ryan D. HESELPOTH, Stamford, CT (US); Raymond SCHUCH, Mountain Lakes, NJ (US); Chad EULER, Forest Hills, NY (US)

(21) Appl. No.: 17/289,878

(22) PCT Filed: Nov. 4, 2019
(86) PCT No.: PCT/US19/59701

§ 371 Date: Apr. 29, 2021

Related U.S. Application Data
(60) Provisional application No. 62/754,898, filed on Nov. 2, 2018.

(51) Int. Cl. C07K 14/21; C12N 9/36; A61P 31/04

(52) U.S. Cl. CPC C07K 14/21; C12N 9/2462; A61K 38/00; C12Y 302/01017; A61P 31/04

(57) ABSTRACT

Provided are polypeptides and compositions comprising the polypeptides for use in killing and/or inhibiting growth of Gram-negative bacteria, particularly P. aeruginosa. The polypeptides are contiguous polypeptides (lysocins) that contain an engineered bacteriocin segment that can be translocated through an outer membrane channel of the Gram-negative bacteria, such as Domain I of the S-type poycin from P. aeruginosa bacterocin pyocin S2 (PyS2) linked to a lysin catalytic segment that has peptidoglycan (PG) hydrolase activity. The lysin catalytic segment can be a catalytic segment of GN4 lysin or any other lysin catalytic segment or a hydrolytic enzyme thereof that has PG hydrolase activity.

### CROSS-REFERENCE TO RELATED APPLICATIONS

This application claims priority to U.S. provisional application No. 62/754,898 filed on Nov. 2, 2018, the disclosure of which is hereby incorporated by reference.

### FIELD

The present disclosure relates to compositions and methods for use in treating Gram-negative bacteria. The compositions comprise recombinant polypeptides referred to herein as “lysocins,” which comprise a segment of a colicin-like bacteriocin, such as an S-type pyocin, and a catalytic segment of a Gram-positive or Gram-negative lysin (lysin) that has peptidoglycan hydrolase activity.

### BACKGROUND

Antimicrobial resistance is a threat to global public health. One of the predominant antibiotic-resistant microorganisms responsible for high mortality rates is Pseudomonas aeruginosa. This Gram-negative pathogen is: i) the leading cause of mortality in cystic fibrosis patients, ii) the main causative agent of burn wound infections, iii) the most frequent Gram-negative bacterium associated with nosocomial and ventilator-acquired pneumonia, and iv) the second most common cause of catheter-associated urinary tract infections (McCaughey L C, et al. Biochem J 473:2345-58.). Additionally, P. aeruginosa is responsible for 3-7% of all bloodstream infections (BSIs) and 23-26% of Gram-negative bacteremias, translating to mortality rates ranging from 27-48% (Hattemer A, et al. 2013. Antimicrob Agents Chemother 57:3969-75.). With the antipseudomonal efficacy of standard of care (SOC) antibiotics progressively diminishing due to a combination of intrinsic, acquired and adaptive resistance mechanisms utilized by the bacteria, the lack of therapeutic options has stimulated the World Health Organization to label P. aeruginosa as a critical priority for the research, discovery and development of new antibiotics.

Bacteriophage (phage)-encoded peptidoglycan (PG) hydrolases, termed lysins, represent an alternative class of antimicrobials to small molecule antibiotics Fischetti Va. 2010. Int J Med Microbiol 300:357-62). During the phage replicative cycle, lysins degrade the PG of host bacteria to induce hypotonic lysis and phage progeny liberation. The extrinsic application of purified recombinant lysins has been validated for antibacterial efficacy towards several Gram-positive bacterial pathogens as a result of the PG constituting part of the exposed outer structural component of the cell. However, expanding the use of these enzymes to target Gram-negative bacteria has, in many cases, been impeded by the outer membrane (OM). To overcome this, lysins have been modified to permit OM translocation. For example, the peptide component of Artilysins, which contain an OM permeabilizing peptide fused to a lysin, locally distorts the lipopolysaccharide layer to allow the lysin to penetrate through the OM (Briers Y, et al. MBio 5:e01379-14). Unfortunately, like most Gram-negative lysins, Artilysins appear to be inactive in human serum (HuS), limiting their therapeutic applicability to superficial, non-systemic bacterial infections (Larpin Y, et al. 2018. PLoS One 13:e0192507; Thandar M, et al. 2016. Antimicrob Agents Chemother 60:2671-9; Briers Y, et al. 2015. Future Microbiol 10:377-90). An alternative engineering strategy has been described using bacteriocin-lysin hybrid molecules to actively transport lysins across protein channels embedded in the OM of Gram-negative bacteria (Yan G, et al. 2017. TAntonie Van Leeuwenhoek 110:1627-1635; Lukacik P, et al. 2012. Proc Natl Acad Sci USA 109:9857-62). For instance, the construction of a lysin-colicin A chimeric molecule yielded a construct (Colicin-Lysep3) capable of traversing the OM of Escherichia coli. Nevertheless, as seen with colicin A, E. coli resistance to Colicin-Lysep3 can seemingly develop by mutating BtuB (the vitamin B12 receptor which also acts as the Colicin-Lysep3 receptor) or OmpF (the pore-forming channel used for Colicin-Lysep3 periplasmic import) (Chai T, Wu V, Foulds J. 1982. J Bacteriol 151:983-8; Cavard D, Lazdunski C. 1981. Fems Microbiology Letters 12:311-316). Notably, E. coli with defective BtuB and OmpF mutations remain virulent (Sampson B A, Gotschlich E C. 1992; Infect Immun 60:3518-22; Hejair H M A, Z et al. Microb Pathog 107:29-37.). There is also no experimental evidence to support Colicin-Lysep3 antibacterial activity in HuS. Thus, there is an ongoing need for improved compositions and methods for treating Gram-negative bacterial infections, which include but are not necessarily limited to P. aeruginosa. The present disclosure is pertinent to this need.

### SUMMARY

The present disclosure provides compositions and methods that relate generally to killing or otherwise inhibiting growth of bacteria, and in particular, Gram-negative bacteria through contact with a contiguous polypeptide that comprises an engineered bacteriocin segment that can be translocated through an outer membrane channel of the Gram-negative bacteria, but is not pesticin or colicin A, linked to a lysin catalytic segment that has PG hydrolase activity, but is not T4 lysozyme (T4L) or Lysep3. In embodiments, the Gram-negative bacteria are present in a bacterial infection in blood, on the skin, the eye, in the cerebral spinal fluid (CSF), the brain, and/or lungs of an individual. In embodiments, the Gram-negative bacteria infection in the lungs of an individual who has cystic fibrosis, or are present in the skin of a burn patient infected with the bacteria, or are in the blood, the eye, CSF or brain of the individual, or a combination thereof. In embodiments, the bacteria are any type of P. aeruginosa. In embodiments, the polypeptide is administered in an amount that is effective to kill the Gram-negative bacteria on the skin, mucosal surfaces, including but not limited to lungs, or in serum, the eye, CSF or brain of the individual. In embodiments, the polypeptide is administered intravenously, topically, intrathecally, or orally, including by inhalation.

In embodiments, the S-type pyocin is P. aeruginosa bacteriocin pyocin S2 (PyS2). In embodiments, the lysin catalytic segment comprises the GN4 lysin or any other lysin catalytic segment or a hydrolytic enzyme thereof that has PG hydrolase activity. In embodiments, the disclosure includes a pharmaceutical formulation comprising a polypeptide with a bacteriocin segment and a lysin catalytic segment, as further described herein.

In non-limiting embodiments, the bacteriocin segment comprises an amino acid sequence that is at least 90% identical to the sequence of amino acids 1-209 of SEQ ID NO:10 (Domain I of PyS2), and wherein the bacteriocin segment does not comprise amino acids 559-689 of SEQ ID NO:10 (Domain IV of PyS2). In embodiments, the bacteriocin segment further comprises an amino acid sequence that is at least 90% identical to the sequence of amino acids 210-312 of SEQ ID NO:10 (Domain II of PyS2). In embodiments, the bacteriocin segment further comprises an amino acid sequence comprising an amino acid sequence that is at least 90% identical to the sequence of amino acids 313-558 of SEQ ID NO:10 (Domain III of PyS2). Thus, in embodiments, each lysocin of this disclosure includes a sequence that is at least 90% identical to the sequence of amino acids 1-209 of SEQ ID NO:10 (Domain I of PyS2), and may further include either or both of an amino acid sequence that is at least 90% identical to the sequence of amino acids 210-312 of SEQ ID NO:10 (Domain II of PyS2), or an amino acid sequence that is at least 90% identical to the sequence of amino acids 313-558 of SEQ ID NO:10 (Domain III of PyS2).

In embodiments, the lysin catalytic segment of the lysocin comprises a segment of a lysin selected from the group of lysins consisting of GN3 lysin, GN4 lysin, PlyG_{cat} lysin, Ply511_{cat} lysin, PlyCd_{cat} lysin, and PlyPa03 lysin. In embodiments, the lysin catalytic segment comprises a GN4 lysin segment that comprises an amino acid sequence that is at least 90% identical to SEQ ID NO:11, and wherein optionally, the first Met of SEQ ID NO:11 is omitted. Thus, in embodiments, the PyS2 segment comprises at least the sequence of amino acids 1-209 of SEQ ID NO:10 and the GN4 lysin segment comprises a sequence that is at least 90% identical of SEQ ID NO:11, wherein optionally, the first Met of SEQ ID NO:11 is omitted.

Contiguous polypeptides as described for use in the methods of the disclosure are included. Expression vectors encoding the contiguous polypeptides are also included, as are methods of making the polypeptides by expressing the expression vector in a suitable cell culture, and optionally separating the polypeptides from the cell culture. The polypeptides may also be purified to any desired degree of purity. Pharmaceutical compositions comprising the polypeptides are also provided, and may be formulated so that they are suitable for use in treating any particular infection, regardless of location, and may also be used for prophylactic purposes, e.g., to prevent or inhibit development of a bacterial infection

### BRIEF DESCRIPTION OF THE FIGURES

FIG. 1. GN4 and PyS2-GN4 purification and antipseudomonal activity. (A) To construct the PyS2-GN4 lysocin, PyS2 domain IV (aa 559-689) was deleted and replaced with the GN4 lysin (aa 1-144). (B) The GN4 lysin (16 kDa) and the wild-type PyS2-GN4 (76 kDa), PyS2-GN4_{ΔTBB} (75 kDa) and PyS2-GN4_{KO} (76 kDa) lysocins were purified to homogeneity according to sodium dodecyl sulfate polyacrylamide gel electrophoresis (SDS-PAGE) analysis. (C) The muralytic activity of purified GN4 and PyS2-GN4 was determined by spotting 25 pmol of each on autoclaved Pseudomonas. Chicken egg white lysozyme (CEWL) and buffer were respectively used as positive and negative controls. Clearing zones signify PG degradation. Using the plate lysis assay, the antipseudomonal activity of (D) GN4, (E) PyS2-GN4, (F) PyS2-GN4_{ΔTBB} and (G) PyS2-GN4_{KO} was determined in growth medium by spotting 0.01-400 pmol purified protein on P. aeruginosa strain 453. (H) The plate lysis assay was further used to analyze the antipseudomonal activity of 0.01-400 pmol PyS2-GN4 against P. aeruginosa strain 453 in 50% HuS. Growth inhibition zones observed using the plate lysis assay indicate antipseudomonal activity.

FIG. 2. PyS2-GN4 killing kinetics and antibiofilm efficacy. The dose-response lysocin killing kinetics were determined by incubating P. aeruginosa strain 453 at 10⁶ colony forming units per milliliter (CFU/ml) statically with 0.01-100 μg/ml PyS2-GN4 for 24 h at 37° C. Bacterial viability was assessed (A) in 2 h increments over the first 12 h in growth medium only and (B) at 24 h with or without HuS. (C) P. aeruginosa strain PAO1 biofilms were grown for 72 h at 37° C. in CAAg medium (casamino acid (CAA) medium with 0.2% (wt/vol) glucose) and subsequently treated for 24 h with buffer or 0.03-500 μg/ml GN4, PyS2-GN4 or tobramycin. The residual biomass of the biofilms was qualitatively measured by means of crystal violet staining. SC represents sterility controls, whereas GC corresponds to growth controls. All error bars correspond to ±standard error of the mean (SEM), while perforated lines mark the limit of detection.

FIG. 3. Visualizing lysocin-treated P. aeruginosa by transmission electron microscopy (TEM). P. aeruginosa strain 453 was incubated with 50 μg/ml PyS2-GN4 in CAA medium with the iron chelator ethylenediamine hydroxyphenylacetic acid (EDDHA) for a total of 1 h at 37° C. The bacteria were then fixed and visualized by TEM at 0, 30 and 60 min post-treatment. Total magnifications of 2,600× (scale bar=1 μm) and 13,000× (scale bar=200 nm) are shown.

FIG. 4. Lysocin cytotoxicity towards eukaryotic cells and bacterial endotoxin release. (A) Human red blood cells (hRBCs) were incubated in triplicate with buffer or 0.5-256 μg/ml PyS2-GN4 for 8 h at 37° C. and hemolysis, as a function of hemoglobin release, was assayed spectrophotometrically at 405 nm. Triton X-100 was used as a positive control for hemolysis. (B) Human promyeloblast HL-60 cells were incubated in triplicate with buffer or 0.5-256 μg/ml PyS2-GN4 for 8 h at 37° C. and viability, as a function of formazan product formation, was measured spectrophotometrically at 570 nm. Triton X-100 was used as a control for cytotoxicity. (C) Endotoxin release was measured in duplicate experiments after treating P. aeruginosa strain 453 at 10⁶ CFU/ml for 1 or 4 h at 37° C. in growth medium with 0.2× and 5×MIC PyS2-GN4, colistin, meropenem or tobramycin. An untreated control was included. All error bars correspond to ±SEM.

FIG. 5. Lysocin antipseudomonal in vivo efficacy using a murine model of bacteremia. Mice (n=100) were intraperitoneally (IP) infected with 10⁸ P. aeruginosa strain 453. (A) The bacterial counts in organs were determined 3 h post-infection in order to confirm the animals were bacteremic. (B) Infected mice were IP treated 3 h post-infection with either buffer (n=35) or 2.5 (n=15), 5 (n=15), 12.5 (n=15) or 25 mg/kg (n=20) lysocin. Survival was monitored over 10 days. All error bars correspond to ±SEM. P-values were calculated using a log-rank (Mantel-Cox) test.

FIG. 6. Schematic overview of the proposed mechanism of PyS2-GN4 antipseudomonal activity. (A) When added extrinsically as a purified recombinant protein, domain I of PyS2-GN4 binds to the ferripyoverdine A type I (FpvAI) receptor located on the surface of target P. aeruginosa. (B) This protein-protein interaction induces a conformational change in the receptor structure, resulting in the FpvAI TonB box (TBB) in the periplasm to recruit and bind TonB1. (C) The formation of this complex allows for the PMF (proton motive force)-dependent unfolding of the labile half of the FpvAI plug domain. (D) Next, the unstructured region of lysocin domain I passes through the channel created in order to enable its own TBB to bind another nearby TonB1 protein in the periplasm. (E) The newly formed lysocin-TonB1 translocon stimulates the PMF-driven unfolding and import of the remainder of the lysocin into the periplasm. (F) Upon refolding, GN4 is proteolytically released and cleaves the PG to provoke partial membrane destabilization, cytoplasmic leakage, PMF disruption and cell death.

FIG. 7. Lysocin thermal stability. PyS2-GN4 was incubated in phosphate buffered saline (PBS) for 30 min at temperatures ranging from 4° C. to 80° C. After cooling on ice, each sample at 50 μg/ml was incubated statically with P. aeruginosa strain 453 for a total of 4 h at 37° C. The average residual antipseudomonal activity of each sample was equated to the log₁₀ decrease in viable bacterial cells when compared to the untreated control. All error bars correspond to ±SEM of triplicate experiments.

FIG. 8. PyS2-I-GN4 construct design, purification and antipseudomonal properties. (A) The parental PyS2-GN4 lysocin consists of PyS2 domains I (aa 1-209), II (aa 210-312) and III (aa 313-558) fused to the GN4 lysin. To construct PyS2-I-GN4, domain I of PyS2 was fused to the GN4 lysin using a GSx3 linker. The linker region of PyS2-I-GN4 was subsequently deleted (PyS2-I-GN4_{NL}) or extended to a GSx6 (PyS2-I-GN4_{12AA}) or GSx9 linker (PyS2-I-GN4_{18AA}). Abbreviations: I, PyS2 domain I; II, PyS2 domain II; III, PyS2 domain III; L, GSGSGS linker. (B) The GN4 lysin (16 kDa) or the PyS2-GN4 (76 kDa), PyS2-I-GN4 (40 kDa), PyS2-I-GN4_{NL} (39 kDa), PyS2-I-GN4_{12AA} (40 kDa) and PyS2-I-GN4_{18AA} lysocins (41 kDa) were purified to homogeneity according to SDS-PAGE analysis. (C) P. aeruginosa strain 453 grown in iron-depleted conditions were incubated with 0.5 μM GN4, PyS2-GN4 or PyS2-I-GN4 at 37° C. for a total of 6 h in iron-chelated CAA medium. (D) P. aeruginosa strain 453, which were initially grown under iron-depleted or iron-rich culture conditions, were incubated in PBS, pH 7.4, with 0.5 μM PyS2-I-GN4 at 37° C. for a total of 6 h. (E) Iron-depleted P. aeruginosa strain 453 cells were incubated with 0.5 μM PyS2-I-GN4_{NL} (NL), PyS2-I-GN4 (6 AA), PyS2-I-GN4_{12AA} (12 AA) or PyS2-I-GN4_{18AA} (18 AA) in iron-chelated CAA medium at 37° C. for a total of 2 h. For each cell viability assay, the CFU/ml concentration of surviving bacterial cells was quantitated by means of serial dilution and plating. Untreated bacteria were used as a negative control for antipseudomonal activity. Limit of detection is 10 CFU/ml. All error bars correspond to ±SEM of triplicate experiments.

FIG. 9. Evaluating the antipseudomonal efficacy of using different lysins for bioengineering lysocins. (A) Additional lysocins were engineered and included domain I of PyS2 fused via a GSx3 linker to either the catalytic domain of the Bacillus anthracis lysin PlyG, the catalytic domain of the Listeria monocytogenes lysin Ply511, the catalytic domain of Clostridium difficile lysin PlyCd, the E. coli lysin T4L, the Pseudomonas putida lysin GN3, or the P. aeruginosa lysin PlyPa03. Abbreviations: I, PyS2 domain I; L, GSGSGS (SEQ ID NO:1) linker. (B) With the exception of (1) PyS2-I-PlyG_{cat} (42 kDa), the (2) PyS2-I-Ply511_{cat} (43 kDa), (3) PyS2-I-PlyCd_{cat} (43 kDa), (4) PyS2-I-T4L (42 kDa), (5) PyS2-I-GN3 (39 kDa) and (6) PyS2-I-PlyPa03 lysocins (40 kDa) were purified to homogeneity according to SDS-PAGE analysis. The additional protein bands observed for the PyS2-I-PlyG_{cat} sample are degradation products. (C) Muralytic activity towards pseudomonal PG was assessed by spotting 25 pmol of each lysocin on autoclaved P. aeruginosa. Clearing zones after 18 h are indicative of muralytic activity. Buffer was spotted as a negative control for muralytic activity, while PyS2-I-GN4 was used as a positive control. (D) The log₁₀-fold killing of P. aeruginosa strain 453 grown in iron-depleted conditions was determined following treatment with 0.5 μM of each lysocin at 37° C. for a total of 4 h in iron-chelated CAA medium. At 1 and 4 h, each sample was serial diluted and plated in order to calculate the CFU/ml concentration of viable bacteria. PyS2-I-GN4 was used as a positive control for bactericidal activity. All data was normalized to the untreated control at each time point. (E) P. aeruginosa strain 453, initially cultured in iron-chelated CAA medium, was incubated with 0.5 μM of PyS2-I-GN3, PyS2-I-GN4 or PyS2-I-PlyPa03 in the presence of 50% beractant (SURVANTA) or 50% HuS at 37° C. for a total of 2 h. Log₁₀-fold killing of P. aeruginosa was quantitated following serially diluting and plating each sample. Each dataset was normalized to the CFU/ml concentration of viable bacteria specific to their respective untreated controls (˜10⁶ CFU/ml for beractant and ˜10⁵ CFU/ml for HuS). Limit of detection is 10 CFU/ml. All error bars correspond to ±SEM of triplicate experiments.

FIG. 10. Comparing the primary amino acid sequence of the PyS2 DNase domain to other lysin candidates utilized for constructing lysocins. Using a multiple sequence alignment by CUSTALW, the amino acid sequence of the PyS2 DNase domain was aligned with the seven different lysins used for bioengineering lysocins. Amino acids shaded black share at least 50% identity between all eight sequences, whereas amino acids shaded grey share at least 50% similarity. PyS2 DNase (SEQ ID NO:2), PlyG_{cat} (SEQ ID NO:3), Ply511_{cat} (SEQ ID NO:4), PlyCd_{cat} (SEQ ID NO:5), T4L (SEQ ID NO:6), GN3 (SEQ ID NO:7), GN4 (SEQ ID NO:8), and PlyPa03 (SEQ ID NO:9).

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all nucleotide and amino acid sequences described herein, and every nucleotide sequence referred to herein includes its complementary DNA sequence, and also includes the RNA equivalents thereof. All sequences described herein, whether nucleotide or amino acid, include sequences having 50.0-99.9% identity, inclusive, and including all numbers and ranges of numbers there between to the first decimal point. The identity may be determined across the entire sequence, or a segment thereof that retains its intended function. Homologous sequences from, for example, other bacteria or bacteriophages as the case may be, are included within the scope of this disclosure, provided such homologous sequences also retain their intended function. Each amino acid sequence and polynucleotide sequence that is described herein by reference to a database is incorporated herein as the sequence exists in the database on the effective filing date of this application or patent. Amino acid sequences that are referred to herein by reference to a database entry, such as by an accession number, are incorporated herein as they exist in the database entry as of the effective filing date of this application or patent.

Any result obtained using a method described herein can be compared to any suitable reference, such as a known value, or a control sample or control value, suitable examples of which will be apparent to those skilled in the art, given the benefit of this disclosure.

The disclosure includes methods of inhibiting growth of bacteria and/or killing bacteria that comprise suitable binding sites to which the recombinant polypeptides described herein attach. “Recombinant” means the polypeptide is made using molecular biology techniques that are known in the art to produce a polypeptide that does not naturally occur in bacteria or bacteriophage. Such techniques include, for example, using any suitable expression vector in any suitable protein expression system. The disclosure includes separating expressing the polypeptides from the expression vector in the expression system, purifying them to any desirable degree of purity, and making compositions including but not necessarily limited to pharmaceutical compositions that contain the polypeptides.

In embodiments the disclosure provides compositions and methods for use in prophylaxis and/or therapy of bacterial infections, and are expected to be suitable for a variety of applications, including but not necessarily limited to treating existing bacterial infections, and to help control antibiotic-resistant infections that may exist at the time compositions of this disclosure are administered, and/or to help limit opportunistic infections that would otherwise establish infections in immunocompromised individuals, or other disorders where bacterial infections are common. Bacteria that are resistant to one or more antibiotics can be killed using embodiments of the disclosure. Likewise, embodiments of this disclosure may be used to synergize the effect of other antimicrobial agents. In embodiments, compositions and methods disclosed herein are for treating infection by P. aeruginosa. In embodiments, prophylactic approaches delivering a composition comprising polypeptides of this disclosure to uninfected individual to prevent development of an infection. In embodiments, a prophylactic approach comprises applying a composition comprising polypeptides of this disclosure to uninfected skin of an individual.

In connection with the present disclosure, phage-encoded PG hydrolases, termed lysins, represent an emerging class of antimicrobials (8). During the phage replicative cycle, lysins degrade host bacterial PG to induce hypotonic lysis and phage progeny liberation. Due to PG accessibility, the extrinsic application of purified recombinant lysins has been validated for antibacterial efficacy towards several Gram-positive bacterial pathogens (9). However, expanding the use of these enzymes to target Gram-negative bacteria has been impeded by the protective OM. It expected that the present disclosure will overcome deficiencies of previously available technologies. In this regard, lysocins can be engineered to kill P. aeruginosa in HuS by exploiting functional domains derived from S-type pyocins, which are chromosomally-encoded colicin-like bacteriocins produced by P. aeruginosa for intraspecies competition (20). These SOS-inducible, high molecular weight proteinaceous toxins are evolutionarily conserved among many Gram-negative bacteria, including Enterobacter cloacae, E. coli, Klebsiella pneumoniae and Yersinia pestis (21). In general, S-type pyocins bind to a ferrisiderophore import receptor located on the target bacterial cell surface. By using the Tol or Ton import system, these bacteriocins actively deliver enzymatic (lipid II degradation, DNase, rRNase, tRNase) or non-enzymatic (inner membrane (IM) pore formation) bactericidal domains to their intracellular targets by translocating across the OM through the channel created by the receptor. These bacteriocin properties are harnessed in embodiments of the present disclosure, as further described below.

In certain aspects, the instant disclosure provides recombinant polypeptides for use as Gram-negative antimicrobial agents. The contiguous polypeptides are referred to herein from time to time as a “lysocin.”

As described further below, the recombinant polypeptides comprise a segment of a colicin-like bacteriocin, such as an S-type pyocin, with the proviso that the bacteriocin is not pesticin or colicin A, and a lysin catalytic segment that has PG hydrolase activity, but is not T4L or Lysep3. In embodiments, the bacteriocin segment and the lysin catalytic segment are configured in the N->C-terminal direction, respectively, but the C->N terminus orientation can also be used. The bacteriocin segment and the lysin catalytic segment may be completely contiguous with one another, or they may be separated by linking amino acids, as described further below. In embodiments, more than one lysin catalytic segment can be included in a polypeptide of this disclosure. Combinations of distinct lysocins and use of such combinations are also included in the disclosure.

In embodiments, the bacteriocin segment comprises any segment of any S-type pyocin that can be transported through an OM channel of a Gram-negative bacteria. All fragments of the S-type pyocin that have this capability are included in this disclosure, with the proviso that pesticin and colicin A can be excluded. In embodiments, the pyocin comprises a single functional domain or multiple functional domains derived from an S-type pyocin, additional characterization and examples of which are provided below. In embodiments, the S-type pyocin can be transported through an OM channel of a Gram-negative bacteria via the Tol and/or Ton import system. In a non-limiting embodiment, and as further described below, the bacteriocin segment is all or a segment of an S-type pyocin produced by P. aeruginosa that is known in the art as pyocin S2 (PyS2).

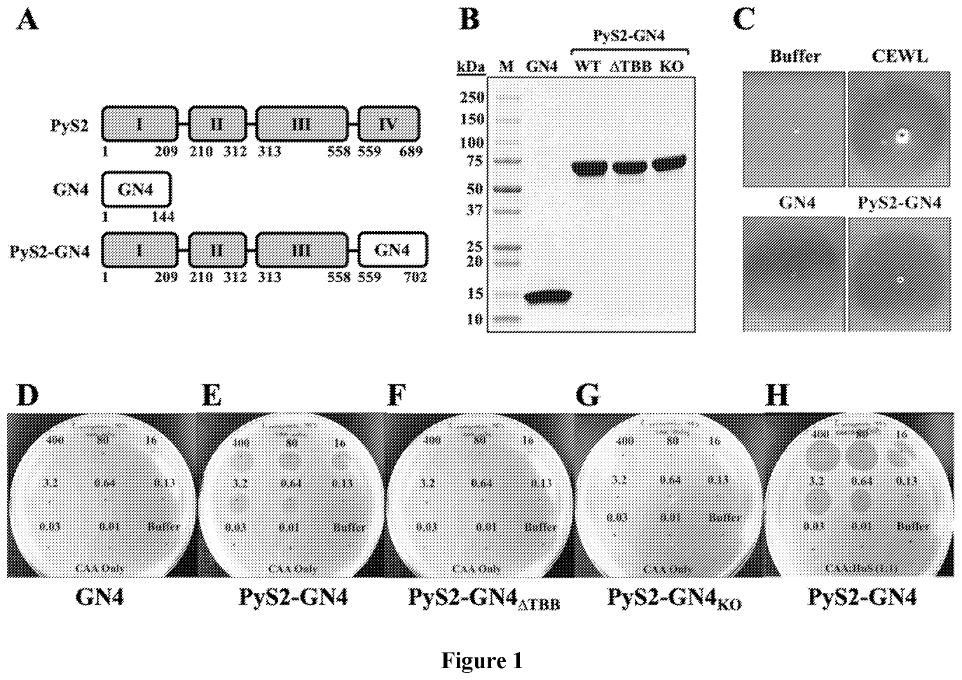

PyS2 contains four domains comprising (I) an N-terminal receptor-binding domain, (II) an α-helical domain, (III) a domain with homology to colicins of E. coli, and (IV) a C-terminal DNase domain (1, 22, 23) (FIG. 1A). Once secreted by the producing cell, PyS2 domain I binds to the ferripyoverdine receptor FpvAI. This gated TonB-dependent transporter is naturally up-regulated in iron-depleted environments to actively import the small siderophore ferripyoverdine. Upon binding PyS2, FpvAI undergoes a structural conformation change. This event results in a short stretch of disordered amino acids encoded by FpvAI, known as the TBB, to periplasmically interact with TonB1, which, along with ExbB and ExbD, is one of three inner IM proteins that constitute the Ton import system (22). The formation of this protein complex stimulates PMF-dependent unfolding of the labile portion of the FpvAI plug domain. Next, the unstructured region of PyS2 domain I (aa 1-45) passes through the newly created channel within FpvAI to present its own TBB (aa 11-15) to another nearby TonB1 protein. Once a PyS2-TonB1 translocon is formed, the PMF drives the unfolding and periplasmic import of the remainder of the bacteriocin. To kill the target cell, PyS2 domain IV refolds, is proteolytically liberated from the remainder of the bacteriocin, and finally translocates through an IM protein channel in order to access its cytosolic deoxyribonucleic acid substrate. Like all S-type pyocin producing strains, P. aeruginosa strains that produce PyS2 also co-express a small immunity protein that transiently binds to and neutralizes the bactericidal domain in order to prevent cellular suicide.

PyS2 has the following amino acid sequence (SEQ ID NO:10):

MAVNDYEPGSMVITHVQGGGRDIIQYIPARSSYGTPPFVPPGPSPYVGTG
MQEYRKLRSTLDKSHSELKKNLKNETLKEVDELKSEAGLPGKAVSANDIR
DEKSIVDALMDAKAKSLKAIEDRPANLYTASDFPQKSESMYQSQLLASRK
FYGEFLDRHMSELAKAYSADIYKAQIAILKQTSQELENKARSLEAEAQRA
AAEVEADYKARKANVEKKVQSELDQAGNALPQLTNPTPEQWLERATQLVT
QAIANKKKLQTANNALIAKAPNALEKQKATYNADLLVDEIASLQARLDKL
NAETARRKEIARQAAIRAANTYAMPANGSVVATAAGRGLIQVAQGAASLA
QAISDAIAVLGRVLASAPSVMAVGFASLTYSSRTAEQWQDQTPDSVRYAL
GMDAAKLGLPPSVNLNAVAKASGTVDLPMRLTNEARGNTTTLSVVSTDGV
SVPKAVPVRMAAYNATTGLYEVTVPSTTAEAPPLILTWTPASPPGNQNPS
STTPVVPKPVPVYEGATLTPVKATPETYPGVITLPEDLIIGFPADSGIKP
IYVMFRDPRDVPGAATGKGQPVSGNWLGAASQGEGAPIPSQIADKLRGKT
FKNWRDFREQFWIAVANDPELSKQFNPGSLAVMRDGGAPYVRESEQAGGR
IKIEIHHKVRIADGGGVYNMGNLVAVTPKRHIEIHKGGK

Representative domains of PyS2 are as follows: Domain I, amino acids 1-209 of SEQ ID NO:10; Domain II: amino acids 210-312 of SEQ ID NO:10; Domain III: amino acids 313-558 of SEQ ID NO:10; Domain IV: amino acids 559-689 of SEQ ID NO:10. In embodiments, at least amino acids 1-21 of domain I are included. In embodiments, at least amino acids 11-15 from domain I of PyS2 are included. In embodiments, the disclosure comprises a contiguous polypeptide that does not include Domain IV of PyS2, which has DNAse activity. Thus, the PyS2, or another S-type pyocin used embodiments of the disclosure, does not have DNAse activity, which can be determined using known methods. In embodiments, the disclosure provides a contiguous polypeptide that includes only Domain I and Domain II, or only Domain I and Domain III, or Domains I, II and III of PyS2, or protein segments that have at least 90% identity to said Domains.

Percent amino acid sequence identity with respect to the polypeptide sequences identified is defined herein as the percentage of amino acid residues in amino acid candidate sequence that are identical with the amino acid residues in given sequences, after aligning the sequences and introducing gaps, if necessary, to achieve the maximum percent sequence identity, and not considering any conservative substitutions as part of the sequence identity. Amino acid sequences of the present disclosure should be considered to include sequences containing conservative changes which do not significantly alter the activity or binding characteristics of the resulting protein. Thus, one of skill in the art, based on a review of the sequence of the amino acid sequences of the polypeptides provided herein and on their knowledge and public information available for other segments of the polypeptides, can make amino acid changes or substitutions in the polypeptide sequences. Amino acid changes can be made to replace or substitute one or more, one or a few, one or several, one to five, one to ten, or such other number of amino acids in the sequence of the polypeptides provided herein to generate mutants or variants thereof. Such mutants or variants thereof may be predicted for function or tested for function or capability for killing bacteria, and/or for having comparable activity to the polypeptides provided herein.

By taking advantage of the molecular characteristics that allow S-type pyocins to traverse the OM, this disclosure provides a strategy for delivering catalytic domains of lysins to their PG substrate in Gram-negative bacteria. In particular, in this disclosure we experimentally validate the in vitro and in vivo antibacterial efficacy of lysocins, which collectively comprise a lysin modified with S-type pyocin functional domains that permit periplasmic import. Thus, in embodiments, the bacteriocin segment is combined with a lysin catalytic segment that has PG hydrolase activity against any pseudomonal PG, with the proviso that the lysin catalytic segment can exclude T4L and Lysep3.

In embodiments, the lysin catalytic segment has enzymatic activity equivalent to an N-acetylmuramidase, lytic transglycosylase, N-acetyl-β-D-glucosamindase, N-acetylmuramoyl-L-alanine amidase, an endopeptidase, or peptidoglycan hydrolase. Catalytic domains of lysins are known in the art, and representative catalytic domains are described below. In embodiments, the catalytic domain is sufficient to exhibit lytic activity against bacteria that are described herein. In embodiments, the lysin catalytic segment comprises all or a functional segment of the lysin known in the art as GN4, which originates from P. aeruginosa PAJU2 phage lysin GN4. In embodiments, GN4 has all or a segment of the following amino acid sequence (SEQ ID NO:11):

MRTSQRGIDLIKSFEGLRLSAYQDSVGVWTIGYGTTRGVTRYMTITVEQA
ERMLSNDIQRFEPELDRLAKVPLNQNQWDALMSFVYNLGAANLASSTLLK
LLNKGDYQGAADQFPRWVNAGGKRLDGLVKRRAAERALFLEPLS

In embodiments, the lysin catalytic segment comprises an amphipathic domain of GN4. In embodiments, the first Met of the GN4 sequence is omitted. In embodiments, the lysin catalytic segment comprises any portion of GN4 described in PCT publication no. WO 2017/049233, published Mar. 23, 2017, the entire disclosure of which is incorporated herein.

In a non-limiting demonstration that is described further below, we engineered a P. aeruginosa-specific lysocin, termed PyS2-GN4, in which domains I to III from PyS2 were fused to the P. aeruginosa PAJU2 phage lysin GN4. Purified PyS2-GN4 was capable of efficiently delivering the lysin to the PG in P. aeruginosa in both the absence and presence of HuS. As a result, the PG was cleaved to stimulate membrane destabilization, cytoplasmic leakage, PMF disruption and bacterial death. Based at least in part on this and other data presented herein, the present disclosure provides for use of the recombinant polypeptides described herein for treating Gram-negative bacterial infections.

In additional non-limiting demonstrations, the disclosure provides lysocins that use only a segment of domain I of PyS2. The disclosure includes comparative data for lysocins that are referred to herein as PyS2-I-PlyG_{cat}, PyS2-I-Ply511_{cat}, PyS2-I-PlyCd_{cat}, PyS2-I-T4L, PyS2-I-GN3, PyS2-I-GN4 and PyS2-I-PlyPa03. Data presented herein indicate that the antipseudomonal killing kinetics of PyS2-I-GN4 and PyS2-I-PlyPa03 are superior to those of PyS2-I-GN3.

Lysocins of this disclosure can be made by adapting conventional molecular biology approaches. For example, DNA sequences encoding any lysocin can be constructed based on the coding sequence of bacteriocins, such as pyocins. Thus, the DNA sequences comprise a sequence encoding a fusion protein that contains segments of the bacteriocin, such as a pyocin. The resulting DNA sequences can be placed into any suitable expression vector. The expression vector can include any additional features that may or may not be part of the encoded fusion proteins, such as any suitable promoter, restriction enzyme recognition sites, selectable markers, detectable markers, origins of replication, etc. The vectors can encode leader sequences, purification tags, and hinge segments that separate two or more other segments of the encoded protein. In an embodiment, the disclosure includes a kit, which may comprise, for example, an expression vector that encodes at least Domain I of PyS2, and a cloning site for introducing a sequence encoding a catalytic fragment of a lysin.

The expression vectors can be introduced into any suitable host cells, which can be prokaryotic cells, or eukaryotic cells. The lysocins can be expressed and separated from cell cultures that produce them using any suitable reagents and approaches, including but not necessarily limited to protein purification methods that use purification tags, including but not limited to histidine tags, and separating the lysocins using such tags. Thus, the disclosure includes isolated polynucleotides encoding the lysocins of this disclosure, cloning intermediates used to make such polynucleotides, expression vectors comprising the polynucleotides that encode the lysocins, cells and cell cultures that comprise the DNA polynucleotides, cells and cell cultures that express the lysocins, their progeny, cell culture media and cell lysates that contain the lysocins, lysocins that are separated from the cells and are optionally purified to any desirable degree of purity, and compositions comprising one or more lysocins. In embodiments, a protein expression system is a prokaryotic expression system.

In certain embodiments a method of the disclosure is implemented using an expression vector, such as a plasmid encoding a suitable lysocin to form a type of DNA vaccine. For example, a composition comprising such an expression vector can be administered instead of, or in addition to, the lysocin(s) themselves. In an embodiment, cells modified to express a lysocin are introduced into a mammal.

In embodiments, the pyocin and lysin domains can be separated from one another using a linker, although data provided herein show that the linker sequence is not required to maintain efficient killing. In embodiments, the pyocin and lysin domains can comprise one or more linkers that connect segments of a single fusion protein, or can connect distinct polypeptides. The term “linker” thus refers to a chemical moiety that connects one segment of a polypeptide to another segment of the same polypeptide, or to another polypeptide, or to another agent. Linkers include amino acids, but other linkers are encompassed as well. Generally speaking, amino acid linkers may be principally composed of relatively small, neutral amino acids, such as Glycine, Serine, and Alanine, and can include multiple copies of a sequence enriched in Glycine and Serine. In embodiments, the linker can comprise from 1-100 amino acids, inclusive, and including all numbers and ranges of numbers there between. In specific and non-limiting embodiments, the linker comprises 3, 4, 5, 6, 7, 8, 9, 10, 11, or 12 amino acids. In a non-limiting embodiment, a GSx3, and thus comprises GS repeated three times. In embodiments, the pyocin and lysin domains are in a contiguous polypeptide in the sequential order of N-terminus-pyocin->lysin-C terminus orientation.

Infections may be treated by using any suitable composition that comprises a pharmaceutically acceptable carrier to thereby provide a pharmaceutical formulation. Thus, in embodiments, one or more lysocins are provided as components of compositions that comprise a pharmaceutically acceptable carrier. The term “pharmaceutically acceptable carrier” as used herein refers to a substantially non-toxic carrier for administration of pharmaceuticals in which the compound will remain stable and bioavailable. Combining a pharmaceutically acceptable carrier in a composition with a lysocin yields “pharmaceutical compositions.” Some suitable examples of pharmaceutically acceptable carriers, as well as excipients and stabilizers can be found in Remington: The Science and Practice of Pharmacy (2005) 21st Edition, Philadelphia, Pa. Lippincott Williams & Wilkins.

In embodiments, therapeutically effective amounts of one or more lysocins are used. Therapeutically effective amount means that amount of a lysocin of this disclosure that will elicit the biological or medical response of a subject that is being sought. In particular, with regard to Gram-negative bacterial infections, the term “effective amount” is intended to include an effective amount of a lysocin of this disclosure that will bring about a biologically meaningful decrease in the amount of or extent of infection of Gram-negative bacteria, including having a bactericidal and/or bacteriostatic effect. The phrase “therapeutically effective amount” is used herein to mean an amount sufficient to prevent, and preferably reduce by at least about 30 percent, more preferably by at least 50 percent, most preferably by at least 90 percent, a clinically significant change in the growth or amount of infectious bacteria. Such changes can be compared to changes in any suitable reference/control. Suitable controls and control values to determine, for example, relative killing activity, will be apparent to those skilled in the art given the benefit of the present disclosure. In embodiments, a lysocin of this disclosure exhibits at least one improved property relative to a control. In embodiments, the control can be any suitable value, such as a property determined from a lysocin with a different binding domain than that in the lysocin under consideration. In embodiments, a lysocin of this disclosure has an improved property relative to a control that at least one of improved inhibition of bacterial growth and/or killing of bacteria, improved protection from the effects of an infection, such as abscess formation, bacteremia, or sepsis, improved reduction in severity of an infection.

Effective amounts of lysocins of this disclosure will depend in part on the duration of exposure of the recipient to the infectious bacteria, the size and weight of the individual, etc.

The duration for use of the composition containing the recombinant polypeptide of this disclosure may also depend on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day.

For any recombinant lysocins disclosed herein, an effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans or non-human animals, such as for veterinary purposes. Precise dosages can be selected in view of the individual to be treated. In certain embodiments, the effective dosage rates or amounts of the polypeptide(s) to be administered, and the duration of treatment will depend in part on the seriousness of the infection, the weight of the patient, the duration of exposure of the recipient to the infectious bacteria, and a variety of a number of other variables. The concentration of the active units or milligrams or micrograms of recombinant polypeptides believed to provide for an effective amount or dosage of enzymes may be selected as appropriate.

Methods of using the therapeutic compositions include administration by any acceptable approaches including but not limited to topically, orally and parenterally. For example, the lysocins can be administered intramuscularly, intrathecally, subdermally, subcutaneously, intravenously, or by aerosol, in any suitable form or formulation. In embodiments, the disclosure comprises direct application of the lysocins using any suitable approaches to directly bring the polypeptide in contact with the site of infection or bacterial colonization, such as to skin, the gastrointestinal tract, mucosa, or application to a wound, as described further below. Compositions comprising lysocins of this disclosure can be directed to the mucosal lining, where, in residence, they kill colonizing disease bacteria. In embodiments, the composition is coated onto or integrated into a substrate, such as a wound dressing, e.g., a bandage.

Due to natural eliminating or cleansing mechanisms of mucosal tissues, conventional dosage forms may not be retained at the application site for a suitable length of time. It may thus be advantageous to have materials, which exhibit adhesion to mucosal tissues, to be administered with one or more lysocins and other complementary agents over a period of time. The disclosure therefore includes use of mucoadhesives, including but not necessarily limited sustained release mucoadhesive and/or bioadhesive formulations, which are known in the art. For compositions requiring absorption in the stomach and upper small intestine and/or topical delivery to these sites, particularly compositions with narrow absorption windows, bioadhesive, and/or gastroretentive drug delivery systems can be used. Compositions requiring absorption or topical delivery only in the small intestine, enteric-coated, bioadhesive drug delivery systems can be utilized. For compositions requiring absorption or topical delivery only in the lower small intestine and colon enteric-coated, bioadhesive drug delivery systems can be utilized.

The forms in which the compositions may be administered include but are not limited to powders, sprays, liquids, ointments, and aerosols. Further, the polypeptides described herein may be in a liquid or gel environment, with the liquid acting as the carrier. A dry anhydrous version of the polypeptide may be administered by an inhaler bronchial spray, although a liquid form of delivery can be used.

The mode of application includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. The polypeptides may be applied to a bandage either directly or in one of the other carriers. The bandages may be sold damp or dry, wherein the polypeptide is in a lyophilized form on the bandage. This method of application is effective for the treatment of infected skin. The carriers of topical compositions may comprise semi-solid and gel-like vehicles that include a polymer thickener, water, preservatives, active surfactants or emulsifiers, antioxidants, and a solvent or mixed solvent system. In embodiments, a composition comprising lysocins described herein can be introduced directly into CSF, or brain.

In embodiments compositions comprising lysocins could be used for coatings of, for example, medical implantable medical devices, and in such situations (which are not exclusive of other situations) may be detectably labelled. In embodiments, lysocins are non-covalently or covalently attached to a substrate. In embodiments one or more lysocins can be attached to a substrate and used in various diagnostic approaches to determine the presence, absence, type and/or amount of bacteria. For example, in certain approaches such polypeptides are reversibly or irreversibly attached to which may be a component of a diagnostic device. Compositions comprising antibodies bound to polypeptides are also included within the scope of this disclosure. In certain approaches the polypeptides described herein are components in an immunological assay, such as for use as a capture or detection agent in, for example, an ELISA assay. In certain approaches the polypeptides are detectably labeled. Any detectable label can be used, non-limiting examples of which include fluorescent labels, labels that can be detected via colorimetric assays, and polypeptides that can produce a detectable signal, such as Green Fluorescent Protein, or any other protein that produces a detectable signal.

In embodiments, the disclosure comprises testing a biological sample from an individual, determining that the individual has a bacterial infection that is suitable for treating with one or more polypeptides described herein, and administering an effective amount of polypeptides described herein to the individual. Any biological sample can be used. Suitable samples include but are not necessarily limited to tissues and biological fluids. In embodiments, the sample comprises blood, urine, saliva, lacrimal secretions, mucosa, esophageal fluid, or any combination thereof. The sample can be obtained using any suitable technique and implement, such as a needle or a swab. The sample can be used directly or can be subjected to a processing step prior to being analyzed.

In certain aspects, the disclosure provides a bacterium or population of bacteria that are in physical association with one or more lysocins of this disclosure.

In certain embodiments, the bacteria to be killed may be on or in an individual, or they can be present on an inanimate surface. In embodiments, the bacteria are present in a biofilm. In embodiments, an infection may be a topical or systemic bacterial infection caused by Gram-negative bacteria. In embodiments, the individual has an infection of blood, and/or eye, and/or CSF, and/or brain, and/or lungs, and/or skin, including but not limited to skin that has been wounded. In embodiments, the wound comprises a burn, such as a burn that comprises tissue damage induced by contact with heated objects and/or surfaces, or light, or chemicals. In embodiments, the wound is caused by medical techniques such as surgical interventions wherein the skin, other tissue or an organ is cut or pierced or avulsed, or other non-medical wounds which cause trauma by any means. In an embodiment, the infection is a catheter-associated urinary tract infection.

In embodiments, the individual is in need of treatment for sepsis, or is at risk for developing sepsis, due to a Gram-negative bacteria infection. In embodiments, the individual has bacteremia.

In embodiments, the individual is in need of treatment for a lung infection. In embodiments, the lung infection is correlated with a lung disorder, including but not necessarily limited to bacterial pneumonia, bronchitis, bronchiolitis, or any acute respiratory infection, chronic obstructive pulmonary disease (COPD), emphysema, and cystic fibrosis. In embodiments, the infection is associated with a nosocomial and/or ventilator-acquired pneumonia. In embodiments, the individual is in need of treatment for any P. aeruginosa infection.

The disclosure is illustrated by Examples provided below. The Examples describe functional domains from a colicin-like bacteriocin fused to a lysin to yield a delivery system that allows the periplasmic import of lysins. The resulting lysocins translocate across the OM of Gram-negative bacteria using Tol- or TonB-dependent transporters to deliver enzymatically-active lysins to their PG substrate, resulting in rapid bacterial death. Considering the ubiquity of colicin-like bacteriocin functional domain and lysin candidates, lysocins can be modified to target numerous Gram-negative bacterial pathogens, based on the present disclosure.

The following examples are presented in order to more fully illustrate the preferred embodiments of the invention and should in no way be construed, however, as limiting the broad scope of the invention.

### Example 1

### GN4 and PyS2-GN4 Muralytic and Antipseudomonal Activity

The amino acid coordinates for the four domains of PyS2 are: domain I (aa 1-209), domain II (aa 210-312), domain III (aa 313-558), and domain IV (aa 559-689) (FIG. 1A). The GN4 lysin, a muramidase from phage PAJU2 of P. aeruginosa, consists of a single globular domain (aa 1-144). To construct the PyS2-GN4 lysocin, domain IV (the DNase domain) was deleted from PyS2 and replaced with the GN4 lysin (FIG. 1A).

To confirm the native GN4 lysin is capable of cleaving pseudomonal PG, purified GN4 (FIG. 1B) was spotted on autoclaved (causing OM disruption) P. aeruginosa, along with CEWL as a control. Clearing zones corresponding to muralytic activity of GN4 (and CEWL) confirmed the lysin can cleave pseudomonal PG (FIG. 1C). Additionally, the purified PyS2-GN4 construct (FIG. 1B) degraded Pseudomonas PG, suggesting GN4 retains its enzymatic activity as a fusion protein (FIG. 1C).

Next, the antipseudomonal activity of GN4 and PyS2-GN4 was investigated in vitro against viable P. aeruginosa. Spotting purified GN4 and PyS2-GN4 on P. aeruginosa lawns revealed GN4 alone was ineffective (FIG. 1D), whereas PyS2-GN4 displayed antipseudomonal activity when applying 0.64 pmol (FIG. 1E). Removing the TBB (aa 11-15) from PyS2-GN4 inhibits antipseudomonal activity by preventing OM translocation (FIG. 1F). This experimental evidence, that a functional lysin can be delivered to the PG of live P. aeruginosa through S-type pyocin fusion, validates the lysocin antimicrobial approach.

The GN4 lysin has a putative active site consisting of a Glu-8aa-Asp-5aa-Thr catalytic triad motif conserved in other lysins that function as glycosylases (21). For PyS2-GN4, these residues are E573, D582 and T588. The purified active site knockout mutant PyS2-GN4_{E573A,D582A,T588A} (PyS2-GN4_{KO}) (FIG. 1B) was incapable of generating distinct growth inhibition zones when applied to P. aeruginosa, indicating PyS2-GN4 antipseudomonal activity is predicated on the muralytic activity of the GN4 lysin (FIG. 1G).

As a first step in evaluating the in vivo therapeutic applicability of lysocins for P. aeruginosa BSIs, the antibacterial properties of PyS2-GN4 were analyzed in HuS. Activity could be observed when spotting 0.13 pmol of lysocin on P. aeruginosa in 50% HuS (FIG. 1H). Compared to growth medium only (FIG. 1E), the increased clarity and diameter of the growth inhibition zones produced by PyS2-GN4 in HuS (FIG. 1H) indicates the antibacterial effect was amplified. This finding could be due to lower free iron availability in serum, which up-regulates the FpvAI receptor.

### Example 2

### Lysocin Killing Kinetics and Antibiofilm Activity

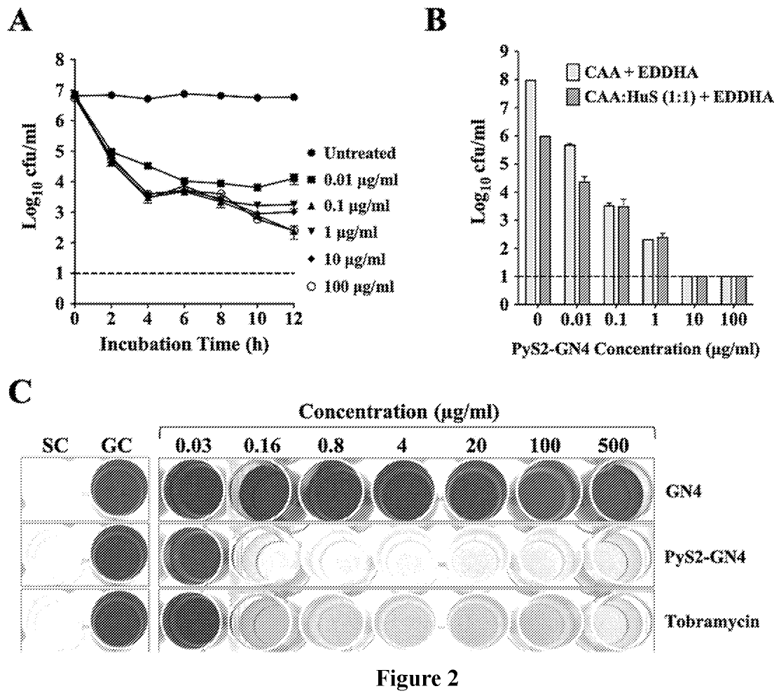

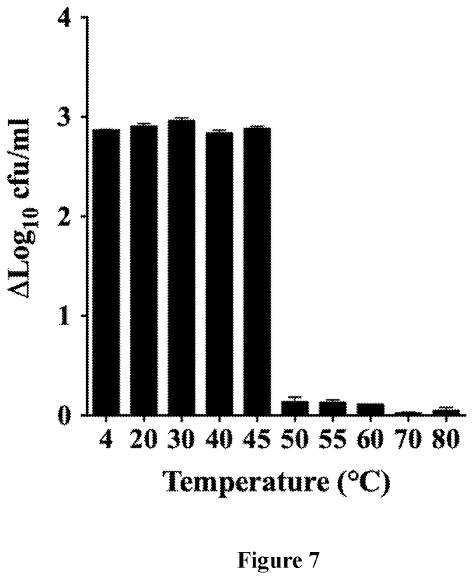

The bactericidal activity of PyS2-GN4 was initially assayed in iron-deficient CAA medium as a function of antimicrobial concentration and time. The medium was supplemented with EDDHA to simulate free iron deprivation in human blood. During the 12 h incubation, 0.1-100 μg/ml lysocin killed P. aeruginosa at a similar rate, resulting in nearly a 2-, 3- and 4-log₁₀ reduction in bacterial viability at 2, 4 and 12 h, respectively (FIG. 2A). The killing kinetics of PyS2-GN4 were considerably reduced when diluted below 0.1 μg/ml. Lysocin concentrations 10 μg/ml (132 nM) sterilized the bacterial culture when incubated in CAA medium and 50% HuS for 24 h (FIG. 2B). In terms of thermal stability, PyS2-GN4 fully retained bactericidal activity following short-term incubation at temperatures 45° C. (FIG. 7). These collective experimental findings indicate PyS2-GN4 is bactericidal at 0.1 μg/ml after 4 h, capable of sterilizing high concentrations of Pseudomonas at nanomolar concentrations in the absence and presence of HuS, and relatively thermostable.

With antibacterial efficacy established in vitro against planktonic P. aeruginosa, the effect of PyS2-GN4 on biofilms was measured. Using a 24-well polystyrene plate, P. aeruginosa biofilms were established for 72 h in CAAg medium and subsequently treated with GN4, PyS2-GN4 or tobramycin for a total of 24 h (FIG. 2C). Residual biofilm biomass was qualitatively assessed by staining with crystal violet. Like planktonic bacteria, the GN4 lysin alone was ineffective against the Pseudomonas biofilm. Alternatively, PyS2-GN4 and tobramycin disrupted biofilm biomass at concentrations ≥0.16 μg/ml. Residual crystal violet observed in the tobramycin 0.16-500 μg/ml treated wells compared to PyS2-GN4 suggests the lysocin is more efficient at degrading biofilms (FIG. 2C).

### Example 3

### Antibacterial Activity Range

The lysocin antibacterial activity range was determined against a collection of P. aeruginosa strains and non-pseudomonal bacteria. Of the 11 P. aeruginosa strains tested, four were lysocin-sensitive (Table 1). PyS2-GN4 displayed minimum inhibitory concentration (MIC) values of ≤4 μg/ml towards the P. aeruginosa reference strain PAO1 and the 452, 453 and MDR-M-3 clinical isolates. As expected, multiplex PCR confirmed these sensitive strains chromosomally encode fpvAI, while the remaining strains encode fpvAII or fpvAIII. Natural resistance to PyS2-GN4 by strains lacking the FpvAI receptor is further evidence lysocin activity is mediated through active transport to the periplasm of sensitive strains. None of the non-pseudomonal bacterial species were lysocin-sensitive (Table 2).

### Example 4

### Benchmarking Lysocin Against SOC Antibiotics

PyS2-GN4 was benchmarked against four SOC antibiotics used clinically for P. aeruginosa BSIs. Using P. aeruginosa strain 453, the MIC and minimum bactericidal concentration (MBC) for PyS2-GN4 were obtained and compared to colistin, meropenem, piperacillin-tazobactam and tobramycin (Table 1). The respective MIC values for PyS2-GN4, colistin, meropenem, piperacillin-tazobactam and tobramycin were 0.25, 0.5, 8, 16 and 0.125 μg/ml. The MBC values for PyS2-GN4, colistin, meropenem, piperacillin-tazobactam and tobramycin were respectively 0.25, 0.5, 8, 128 and 0.25 μg/ml.

### Example 5

### Visualizing the Mechanism of PyS2-GN4 Antipseudomonal Activity

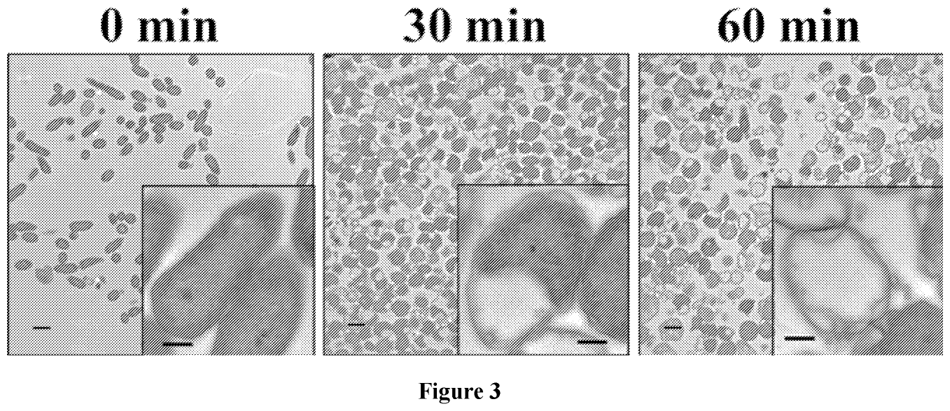

P. aeruginosa treated with lysocin were visualized by TEM to better understand the mechanism of PyS2-GN4 antipseudomonal activity (FIG. 3). Untreated bacteria were rod-shaped with uniform intracellular density. Conversely, P. aeruginosa at 30 min post-lysocin treatment transitioned from rod-shaped to a more spherical morphology. This phenotype is indicative of bacteria with a defective cell wall; visual evidence of GN4 muralytic activity. Furthermore, by cleaving the PG, the integrity of the OM and IM appears to be partially compromised through hypotonic pressure, resulting in cytoplasmic leakage and PMF disruption. At 60 min post-lysocin treatment, a significant portion of the bacterial population are intact, nonviable cells either lacking or with noticeably reduced cytoplasmic content.

### Example 6

### Lysocin Cytotoxicity

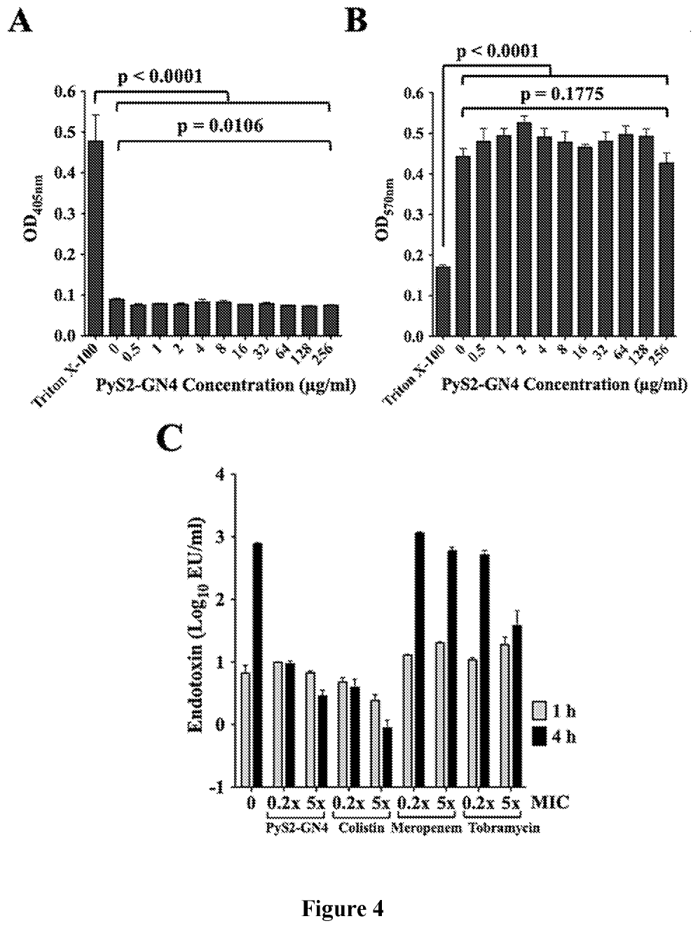

Lysocin cytotoxicity was initially measured using two different eukaryotic cell types. hRBCs (FIG. 4A) and human promyeloblast HL-60 cells (FIG. 4B) were incubated with 0.5-256 μg/ml PyS2-GN4 for 8 h. Contrary to the Triton X-100 controls, no cytotoxicity was observed in the presence of lysocin. Next, endotoxin release was measured in growth medium after treating P. aeruginosa with lysocin or SOC antibiotics (FIG. 4C). Compared to the 1 h time point, the increase in endotoxin detected for the untreated control at 4 h may be attributed to cell division events (22). Endotoxin release stimulated by PyS2-GN4 and colistin, which has potent anti-endotoxin activity (23), was approximately 100- to 1,000-fold less than meropenem and tobramycin after 4 h treatment. Unlike colistin, which binds and neutralizes liberated endotoxin, the low amount of endotoxin detected in the lysocin-treated samples relates to the ability of PyS2-GN4 to kill Pseudomonas with minimal disruption of the OM (FIG. 3), allowing endotoxin to remain anchored to the bacterial surface.

### Example 7

### In Vivo Antipseudomonal Efficacy Using a Murine Model of Bacteremia

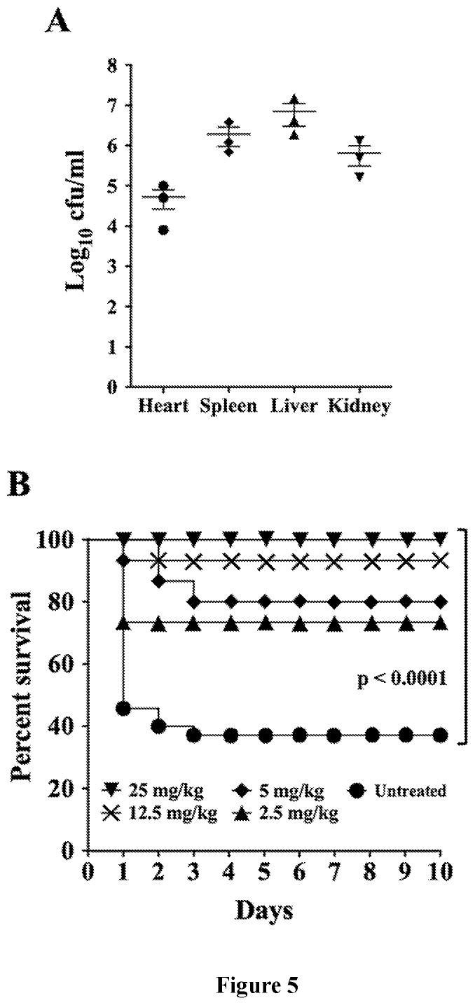

The in vivo antipseudomonal efficacy of PyS2-GN4 was examined using a murine model of bacteremia. Mice were injected IP with P. aeruginosa strain 453 and then treated IP 3 h post-infection with various doses of lysocin; survival was monitored for 10 days. At 3 h post-infection, mice were bacteremic, with bacterial concentrations in the heart, spleen, liver and kidney ranging from ˜10⁴-10⁶ CFU/ml (FIG. 5A). In this model, only 37% of buffer-treated control animals survived the duration of the experiment (FIG. 5B). Alternatively, when mice were treated with 2.5, 5, 12.5 and 25 mg/kg lysocin, 73%, 80%, 93% and 100% were respectively protected from death. Organs of surviving lysocin-treated mice did not contain detectable Pseudomonas at day 10 (data not shown).

### Example 8

### Methods

### Bacterial Strains and Culture Conditions

The bacterial strains used in this disclosure are outlined in Table 3. P. aeruginosa strains numbered 443-453 were clinical isolates from the clinical laboratory of Weill Cornell Medical Center (New York, N.Y.). Further details relating to the site of isolation and clinical disease are unavailable. P. aeruginosa strain MDR-M-3, a multi-drug resistant clinical isolate originating from the lungs of a patient with cystic fibrosis, was obtained from Columbia University Medical Center (New York, N.Y.). All Gram-negative strains were routinely grown in Luria-Bertani (LB) or CAA medium (5 g/L casamino acids, 5.2 mM K₂HPO₄, 1 mM MgSO₄). Gram-positive strains were grown in trypticase soy broth (Bacillus cereus and Staphylococcus aureus), Brain Heart Infusion (BHI) broth (Enterococcus faecium), or Todd Hewitt broth with 1% (wt/vol) yeast extract (Streptococcus pyogenes).

### Molecular Cloning and Mutagenesis

Genes encoding translated GN4 (YP_002284361) and PyS2 (NP_249841) were synthesized and codon-optimized for protein expression in E. coli (GeneWiz, Inc.). The gn4 and pys2-gn4 genes were cloned into the E. coli expression vector pET28a using the NEBuilder HiFi DNA Assembly Cloning Kit (New England Biolabs). pys2 nucleotides 1-1,674 and gn4 were amplified using the polymerase chain reaction (PCR). The 50 μl PCR mixture consisted of 1 ng template DNA, 1×Q5 Reaction Buffer, 0.2 mM dNTPs, 0.5 μM of each oligonucleotide primer, and 1 U of Q5 DNA polymerase (New England Biolabs). The primers used to amplify gn4 (GN4° F./GN4R),pys2 fragment of pys2-gn4 (PyS2_F/PyS2-GN4_R) and gn4 fragment of pys2-gn4 (PyS2-GN4_F/GN4_R) are listed in Table 4. The thermocycler heating conditions were 98° C. for 30 s, 35× (98° C. for 10 s, 60° C. for 30 s, 72° C. for 30 s/kb) and 72° C. for 2 min. Next, a 20 μl reaction consisting of 1× NEBuilder HiFi DNA Assembly Master Mix, gn4 or pys2 gn4 PCR product(s), and NcoI/BamHI linearized pET28a was incubated at 50° C. for 15 min and transformed into E. coli DH5a. Following sequence confirmation, pET28a::gn4 and pET28a::pys2-gn4 were transformed into E. coli BL21(DE3).

PyS2-GN4_{ΔTBB} was created by amplifying pET28a::pys2-gn4 with phosphorylated primers bordering pys2-gn4 nucleotides 33-45. PyS2-GN4_{E573A,D582A,T588A} (PyS2-GN4_{KO}) was generated using two sequential site-directed mutagenesis reactions. Each 50 μl PCR mixture consisted of 50 ng template DNA, 1×Q5 Reaction Buffer, 0.2 mM dNTPs, 0.5 μM of each oligonucleotide primer (Table 4), and 1 U of Q5 DNA polymerase. For the TBB deletion mutant, pET28a::pys2-gn4 was amplified with PyS2-GN4ΔTBB_F/PyS2-GN4ΔTBB_R to create pET28a::pys2-gn4_{ΔTBB}. For the active site mutant, pET28a::pys2-gn4 was initially amplified with PyS2-GN4_KO_1F/PyS2-GN4_KO_1R to generate pET28a::pys2-gn4_{E573A,D582A}. Next, pET28a::pyS2-gn4_{E573A,D582A} was amplified with PyS2-GN4_KO_2F/PyS2-GN4_KO_2R to obtain pET28a::pys2-gn4_{KO}. The thermocycler heating conditions were 98° C. for 30 s, 25× (98° C. for 10 s, 60° C. for 30 s, 72° C. for 30 s/kb) and 72° C. for 2 min. The PCR products were ligated using T4 DNA ligase (New England Biolabs) and transformed into E. coli DH5a. Following sequence confirmation, pET28a::pys2-gn4_{Δ7BB} and pET28a::pys2-gn4_{KO} were transformed into E. coli BL21(DE3).

### Protein Expression and Purification

Using E. coli BL21(DE3), GN4, PyS2-GN4, PyS2-GN4_{ΔTBB} and PyS2-GN4_{KO} were expressed for 4 h at 37° C. with shaking at 200 RPM in LB containing 50 μg/ml kanamycin. Protein expression was induced at mid-log phase (OD₆₀₀=0.5) with 1 mM isopropyl β-D-1-thiogalactopyranoside. The cells were then harvested, washed, resuspended in 50 mM Tris-HCl, pH 7.5, 200 mM NaCl, 1 mM phenylmethanesulfonyl fluoride (PMSF), and lysed using an Emulsiflex-C5 homogenizer (Avestin). The lysate was cleared by centrifugation at 13,000 RPM for 1 h at 4° C. The soluble lysate fraction was dialyzed against 10 mM sodium phosphate, pH 7.0, followed by sterile filtration (0.2 μm) to generate the crude lysate.

The crude lysate was applied to a HiTrap CM FF column (GE Healthcare Life Sciences) in 10 mM sodium phosphate, pH 7.0, using an AKTA fast protein liquid chromatography (FPLC) system (GE Healthcare Life Sciences). Protein was eluted from the column using a linear gradient from 0 to 250 mM NaCl. Elution fractions containing the protein of interest were dialyzed against 50 mM Tris-HCl, pH 7.5, 200 mM NaCl, and concentrated using an Amicon Ultra Ultracel-10K (GN4) or -50K (lysocin) filter (EMD Millipore). The protein samples were then applied to either a HiLoad 16/60 Superdex 75 (GN4) or 200 (lysocin) Prep Grade column (GE Healthcare Life Sciences) in 50 mM Tris-HCl, pH 7.5, 200 mM NaCl. Highly pure GN4 and lysocin elution fractions were combined, concentrated, sterile filtered and stored at −80° C. until further needed.

### Plate Lysis Assay Using Autoclaved or Viable Pseudomonas

For determining muralytic activity, 25 pmol of each purified protein sample was spotted on 0.75% (wt/vol) agarose embedded with autoclaved P. aeruginosa in 50 mM Tris-HCl, pH 7.5. Clearing zones observed after 24 h incubation at 37° C. correspond to muralytic activity. For elucidating antipseudomonal activity towards viable bacteria, 0.01-400 pmol of each purified protein sample was spotted on 0.75% (wt/vol) agarose comprising P. aeruginonsa strain 453 at an initial concentration of 5×10⁶ CFU/ml in either CAA medium or CAA:HuS (1:1; HuS from pooled human male AB plasma, Sigma-Aldrich). Growth inhibition zones observed after 24 h incubation at 37° C. correspond to antipseudomonal activity. Buffer was spotted as a negative control.

### Dose-Response Cell Viability Assay

For the 12 h experiments, 0.01-100 μg/ml of PyS2-GN4 was incubated statically at 37° C. with P. aeruginosa strain 453 at 10⁶ CFU/ml in CAA medium with 0.5 mg/ml EDDHA (Complete Green Company). At 2 h increments, an aliquot was removed from each sample, serial diluted and plated on CAA agar to determine viable bacterial counts. For the 24 h experiments, lysocin at 0.1-100 μg/ml was incubated statically at 37° C. with P. aeruginosa strain 453 at 10⁶ CFU/ml in either CAA medium or CAA:HuS (1:1) with EDDHA. After 24 h, the samples were serial diluted and plated on CAA agar to assess bacterial viability. An untreated control was used for each data set. All samples were investigated in duplicate.

### Biofilm Disruption Assay

The biofilm disruption assay was modified from a previously described method (43). Wells of a 24-well flat-bottom polystyrene tissue culture plate were inoculated with P. aeruginosa strain PAO1 at 5×10⁵ CFU/ml in 2 ml CAAg medium. Sterility controls consisting of growth medium only were included. Biofilms were grown at 37° C. for 72 h with humidity at 120 RPM. Biofilms were washed twice with PBS and treated statically for 24 h with buffer or antimicrobial at 0.03-500 μg/ml in 2.5 ml CAAg supplemented with EDDHA. After treatment, each well was washed twice, stained for 10 min with 0.05% (wt/vol) crystal violet, and washed an additional three times. To qualitatively measure biofilm biomass, residual crystal violet stain in each well was solubilized with 2 ml 33% (vol/vol) glacial acetic acid and imaged. Each sample was analyzed in duplicate.

### Multiplex PCR to Determine FpvA Receptor Type

As previously described, six oligonucleotide primers were used for the simultaneous amplification of different fpvA gene types (44). fpvAI- (326 bp), fpvAII- (897 bp) and fpvAIII-specific (506 bp) gene fragments were respectively amplified with FpvAI_F/FpvAI_R, FpvAII_F/FpvAII_R and FpvAIII_F/FpvAIII_R primers (Table 4). The 25 μl multiplex PCR reaction consisted of 0.2 mM dNTPs, 0.5 μM of each oligonucleotide primer, 1× Taq Reaction Buffer, 1 U of Taq DNA polymerase (New England Biolabs), and 1 μl of an overnight P. aeruginosa culture. The thermocycler heating conditions were 95° C. for 5 min, 35× (95° C. for 30 s, 55° C. for 30 s, 68° C. for 30 s/kb) and 68° C. for 10 min.

### Measuring MIC and MBC

The MIC and MBC values were calculated using a modified version of the broth microdilution assay as previously described by the Clinical and Laboratory Standards Institute (CLSI) (45). The specific modifications were to the bacterial concentration and growth medium used. Briefly, using a 96-well flat-bottomed microtiter plate, bacteria at 10⁴ CFU/ml were incubated statically in triplicate with 0.002 to 256 μg/ml antimicrobial in either Mueller Hinton Broth (MHB; Gram-positive bacteria) or CAA medium (Gram-negative bacteria) for 48 h at 37° C. CAA medium was used for Gram-negative bacteria to simulate low iron conditions. Alternatively, MHB was used for Gram-positive bacteria due to their inability to grow in CAA medium. Bacterial growth was assessed by measuring the OD_{600 nm} using a SpectraMax M5 microplate reader (Molecular Devices). The MIC was defined as the lowest antimicrobial concentration that inhibits bacterial growth. To determine the MBC, the contents from each well originating from the MIC microtiter plate was plated on CAA agar to quantitate bacterial viability. The MBC was defined as the lowest antimicrobial concentration required to kill ≥99.9% of the initial bacterial inoculum. Growth and sterility controls were included.

### Transmission Electron Microscopy

P. aeruginosa strain 453 at 10⁸ CFU/ml was incubated statically with lysocin at 50 μg/ml in CAA medium with EDDHA for a total of 1 h at 37° C. At 0, 30 and 60 min, an aliquot was removed and fixed with 100 mM sodium cacodylate, pH 7.4, containing 4% (vol/vol) paraformaldehyde and 2% (vol/vol) glutaraldehyde. TEM images were obtained by The Rockefeller University Electron Microscopy Resource Center.

### Cytotoxicity Assays

For the hemolytic assays, blood was obtained from healthy adult donors. This study was approved by our Institutional Review Board and all adult subjects provided a written informed consent. In this assay, human blood was initially collected in an EDTA-containing conical tube was obtained from The Rockefeller University Hospital. hRBCs were harvested by centrifugation at 800×g for 10 min, washed three times with PBS, and resuspended in buffer to a 10% (vol/vol) concentration. Next, using a 96-well flat-bottomed microtiter plate, 100 μl hRBC solution was mixed 1:1 in triplicate with a final concentration of 0.5 to 256 μg/ml PyS2-GN4 in buffer. PBS with or without 0.01% (vol/vol) Triton X-100 were used as positive and negative controls for hemolysis, respectively. The microtiter plate was incubated for 8 h at 37° C. Intact hRBCs were removed by centrifugation. To quantitate the relative concentration of hemoglobin release, 100 μl of the sample supernatant was transferred to a new 96-well microtiter plate and the absorbance was measured at an OD_{405 nm} using the microplate reader.

Cytotoxicity towards the human promyeloblast HL-60 cells was determined using the CellTiter 96 Non-Radioactive Cell Proliferation Assay (Promega). Briefly, HL-60 cells (ATCC CCL-240) were harvested at 1,500 RPM for 5 min, washed twice with PBS, and resuspended to a concentration of 2×10⁶ viable cells/ml based on Trypan Blue exclusion tests. Using a 96-well flat-bottomed microtiter plate, 1×10⁵ HL-60 cells were mixed in triplicate with a final concentration of 0.5 to 256 μg/ml PyS2-GN4. As a positive and negative control for cytotoxicity, HL-60 cells were incubated in PBS with or without 0.01% Triton X-100, respectively. The samples were incubated for 8 h at 37° C. with 5% CO₂. Next, the Dye Solution was added to each sample. Viable cells convert the tetrazolium component of the Dye Solution into a formazan product. The microtiter plate was incubated for another 4 h. Solubilization/Stop Solution, which solubilizes the formazan product, was then added and the plate was further incubated overnight at 37° C. The relative amount of formazan product was measured at an OD_{570 nm} using the microplate reader.

Endotoxin Release P. aeruginosa strain 453 at 10⁶ CFU/ml was treated with 0.2× and 5×MIC PyS2-GN4, colistin, meropenem or tobramycin in CAA medium for either 1 or 4 h at 37° C. The samples were subsequently centrifuged at 2,000×g for 10 min. The supernatant was collected and passed through a 0.2 μm syringe filter. Endotoxin concentration in the filtered supernatant was quantitated using the ToxinSensor Chromogenic LAL Endotoxin Assay Kit (GenScript). All data was depicted as the mean±SEM of duplicate experiments.

### Murine Model of Bacteremia

A murine model of bacteremia using P. aeruginosa was adapted from previous studies (46-50). Briefly, male 6-week-old C57BL/6 mice (Charles River Laboratories) were IP infected with 10⁸ P. aeruginosa strain 453 and then IP treated 3 h post-infection with a single dosage of either PBS or PyS2-GN4 at 2.5-25 mg/kg. Survival was monitored for 10 days. The collective results were obtained from four independent experiments and analyzed by Kaplan-Meier survival curves using GraphPad Prism. The Rockefeller University Institutional Animal Care and Use Committee approved all mouse experiments (protocol 17025).

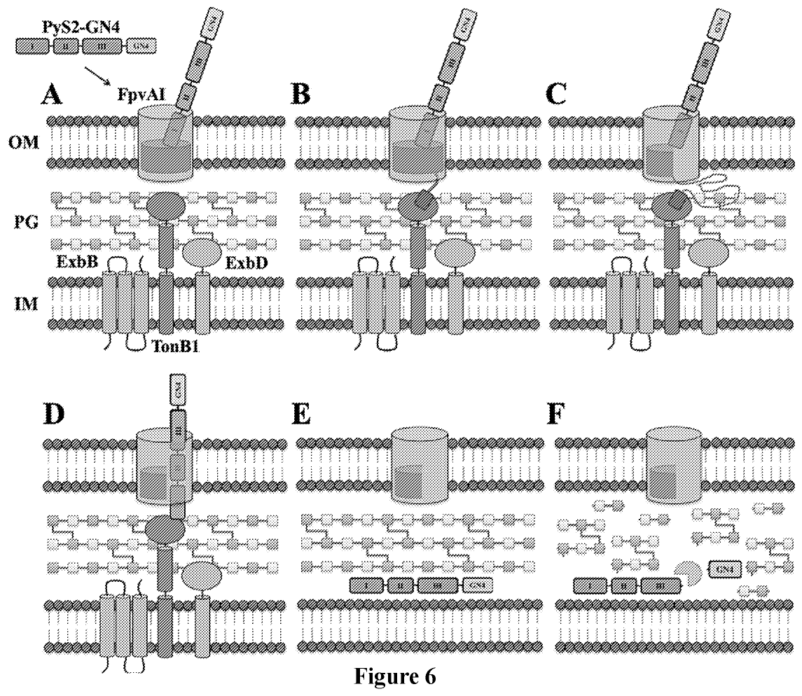

It will be recognized from the foregoing that the present disclosure provides experimentally validated in vitro and in vivo the use of lysocins, which represent a class of bioengineered antimicrobials that deliver phage lysins to their PG substrate in Gram-negative bacteria. In one approach, the P. aeruginosa-specific PyS2-GN4 lysocin was designed by fusing PyS2 domains I-III to the GN4 lysin. With an understanding of the molecular characteristics associated with colicin-like bacteriocins and lysins, and without intending to be bound by any particular theory, a non-limiting model describing a proposed mechanism of PyS2-GN4 antipseudomonal activity is provided based on results presented in this disclosure (FIG. 6). Without intending to be bound by any particular theory, it is considered first that the lysocin targets P. aeruginosa due to domain I binding with high specificity to FpvAI (FIG. 6A). This interaction induces a conformational change in the receptor structure, allowing the FpvAI TBB to interact with TonB1 in the periplasm (FIG. 6B). The formation of this complex causes the PMF-driven unfolding (and opening) of the labile half of the FpvAI plug domain (FIG. 6C). The N-terminal unstructured region of the lysocin passes through the newly created opening to allow its own TBB to form a translocon with TonB1 (FIG. 6D). The PMF energizes the remainder of PyS2-GN4 to unfold and translocate into the periplasm, where the protein subsequently refolds (FIG. 6E). It is believed the GN4 lysin is putatively proteolytically liberated and cleaves the PG through hydrolysis of the 13-1,4 glycosidic bond between N-acetylmuramic acid and N-acetylglucosamine (FIG. 6F). Due to cytoplasmic pressure, loss of PG structural integrity rapidly promotes membrane destabilization, cytoplasmic leakage and PMF disruption, thereby killing the bacterial cell.

The receptor bound by domain I of the pyocin is limited to certain bacterial species and strains. The lysocin approach is therefore narrow spectrum, with minimal effects on the normal microflora. This is supported in vitro by the inability of PyS2-GN4 to kill P. aeruginosa strains lacking the FpvAI receptor (Table 1) and non-pseudomonal bacterial species (Table 2), significantly reducing the possibility of antibacterial activity on bystander commensal microorganisms in vivo.

Like PyS2-GN4, several potential pyocin-related lysocins appear to bind and translocate through receptors involved in ferrisiderophore import. Expression of these receptors is inversely regulated by free iron availability. Considering free iron concentration in HuS is ˜10′⁴M, Pseudomonas in the bloodstream would be highly susceptible to the presently provided lysocins due to receptor up-regulation stimulated by free iron depletion; as exemplified in FIG. 1H, 2B, 5B (24). This highlights the therapeutic potential of lysocins as narrow-spectrum antimicrobials for P. aeruginosa.

In addition to their potency towards planktonic bacteria, lysocins can potentially be used to breakdown pseudomonal biofilms. Mucoid P. aeruginosa biofilms are a major cause of morbidity and mortality in cystic fibrosis patients because of their ability to promote chronic lung infections (25). The physical barrier of biofilms permits constituent bacterial cells to resist immune cell opsonization and phagocytosis, while also increasing tolerance to toxic oxygen radicals and antibiotics (26). Non-limiting demonstrations of this disclosure provide evidence that lysocins may be used as effective antibiofilm agents (FIG. 2C).

Antibiotic-mediated endotoxin release during treatment of Pseudomonas bacteremia can have immediate adverse effects on patient morbidity. Once released, the endotoxin lipid A moiety stimulates immune cells to secrete proinflammatory cytokines, promoting endothelial damage and severe hemodynamic and metabolic disorders (27). As depicted in FIG. 4C, compared to SOC antibiotics meropenem and tobramycin, lysocin-treated P. aeruginosa released ˜100-fold less endotoxin after 4 h. Besides translocating into the periplasm without perturbing the OM, lysocins of this disclosure employ an antibacterial mechanism that kills bacteria while simultaneously preventing destructive cell lysis; keeping endotoxin anchored to the intact OM of the nonviable cells (FIG. 3, 6). This feature is encompassed by the disclosure.

PyS2 functional domains were exploited for these non-limiting demonstrations, as this bacteriocin was one of the first S-type pyocins discovered (19, 20, 28-34). With the lysocin methodology validated, desired properties can be strategically engineered to improve therapeutic applicability. For example, PyS2-GN4 has strain specificity conferred by domain I, which binds FpvAI. The three P. aeruginosa FpvA receptor types are FpvAI, FpvAII and FpvAIII, and each are equally distributed among clinical isolate populations, suggesting one-third of clinically-relevant P. aeruginosa strains will be sensitive to PyS2-GN4 (35). This indicates that while PyS2-GN4 may be used alone, in view of the present disclosure, different strategies can be used to broaden strain coverage. In non-limiting embodiments, lysocins can be constructed with pyocin receptor-binding domains that recognize more conserved receptors. For instance, the receptor-binding domain of pyocin S5 binds the highly conserved ferripyochelin FptA receptor and demonstrates species-specific bactericidal activity, with the exception of strains naturally expressing this pyocin and its immunity protein (36). Strain coverage can be also expanded by formulating lysocin cocktails that bind all three FpvA receptors or by fusing multiple unique receptor-binding domains together.

Natural resistance has hindered development of S-type pyocins clinically as antimicrobial agents. P. aeruginosa are genetically programmed to express an immunity protein that renders the bacterium insusceptible to the bactericidal effects of any chromosomally-encoded S-type pyocins, whether produced inherently or by neighboring Pseudomonas. This is circumvented when constructing lysocins, since the C-terminal bactericidal domain of the pyocin targeted by the immunity protein is replaced with a lysin. The binding specificity of immunity proteins prevent recognition and neutralization of the lysin component of lysocins. Consequently, P. aeruginosa intrinsically resistant to the parental pyocin will be vulnerable to the lysocin.

An attempt was made to investigate the ability of P. aeruginosa to develop resistance to PyS2-GN4 using serial passage experiments under iron-depleted growth conditions. The experimental design included initially determining the MIC of the lysocin in iron-chelated CAA medium. Pseudomonas growing at the highest lysocin concentration were to be used in a subsequent MIC assay; this process was to be repeated at least 15 times. However, the bacteria were incapable of growing with or without lysocin under these iron-depleted conditions. Although acquired resistance to native pyocins is often attributed to chromosomal alterations that generate defective or down-regulated FpvA receptors, thus inhibiting import of FpvA-dependent lysocins, obstructing the ferripyoverdine import system in Pseudomonas would result in avirulent strains (29, 38-40). Lysocin translocation can alternatively be impeded by modifying components of Tol or Ton import systems. However, inactivating TolA or TolQ of the Tol system was proposed to be lethal in P. aeruginosa, while TonB mutants are avirulent and incapable of growing in iron-depleted environments due to their inability to acquire iron mediated by pyoverdin, pyochelin and heme uptake (41, 42). A third potential resistance mechanism involves mutating the chemical composition of PG to inhibit lysin muralytic activity. Lysin resistance has not been observed to date, which is attributable to phage coevolving with their bacterial hosts over millions of years. This has resulted in evolving lytic enzymes that cleave conserved and immutable targets in the PG, making resistance formation a very rare event.

Besides Colicin-Lysep3 and the presently provided PyS2-GN4, another bacteriocin-lysin hybrid molecule has been described. Pesticin, a colicin-like bacteriocin that targets Y. pestis and uropathogenic E. coli, contains a bactericidal domain that naturally functions as a lysozyme-like muramidase. By replacing this domain with T4L, the resulting hybrid molecule transported T4L to the periplasm of E. coli (12). T4L is structurally superimposable and functionally identical to the pesticin muramidase domain. A characteristic differentiating PyS2-GN4 from Colicin-Lysep3 and the pesticin hybrid molecule is that the lysocin retains bactericidal activity in HuS. There is no evidence to support activity in serum for the other two hybrid molecules.

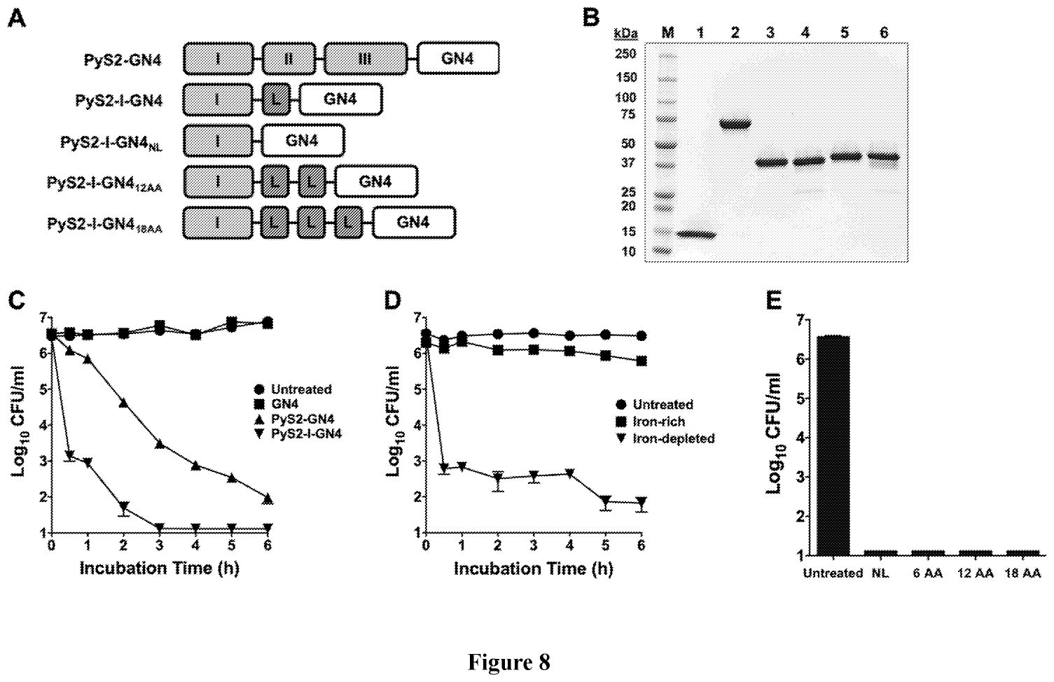

In a related approach, deleting domains II and III from PyS2-GN4 to generate a truncated lysocin construct, termed PyS2-I-GN4 (FIGS. 8A and 8B), enhanced bactericidal activity (FIG. 8C). This finding illustrates that lysins can be transported across the OM of target bacteria solely using the component(s) of colicin-like bacteriocins directly responsible for receptor-binding and Tol/TonB-mediated import. It is considered, without intending to be bound by any particular concept, that the improvement in antipseudomonal potency of PyS2-I-GN4 can be attributed to the reduced size of the truncated lysocin (40 kDa) compared to full-length PyS2-GN4 (76 kDa). PyS2-I-GN4 may require less time and energy than PyS2-GN4 for both TonB1-mediated unfolding during OM translocation, as well as refolding by periplasmic chaperones. As such, the efficiency of OM translocation for PyS2-I-GN4 would be greater than that of the full-length lysocin, resulting in a more rapid accumulation of lysocin molecules in the periplasm over time.

Similar to the parental lysocin, the antipseudomonal activity displayed by PyS2-I-GN4 is influenced by FpvAI expression as a function of iron availability (FIG. 8D). In iron-replete conditions, pvd (responsible for pyoverdine biosynthesis) and fpvA (encodes the ferripyoverdine type A receptor) are not transcribed. This is due to the ferric uptake regulator (Fur) repressing transcription of the regulatory genes pvdS (sigma factor that directs transcription of pvd), fpvI (sigma factor that controls transcription of fpvA) and fpvR (an anti-sigma factor for PvdS and FpvI) (3, 4). In iron-limiting conditions (<1 μM), Fur repression is relieved, allowing for transcription of pvdS, fpvI and fpvR (3, 5-8). A subsequent signaling cascade prompted by extracellular pyoverdine binding FpvA ultimately prevents the anti-sigma factor FpvR from antagonizing PvdS and FpvI, which in turn allows the two proteins to activate transcription of pvd and fpvA, respectively (7, 8). Because of its toxicity, free iron in the human body is maintained at low concentrations under physiological conditions. Limited iron availability combined with the requirement of pyoverdine production for virulence (9) indicates pathogenic P. aeruginosa would be susceptible to FpvA-targeting lysocins due to the bacteria actively expressing the receptor.

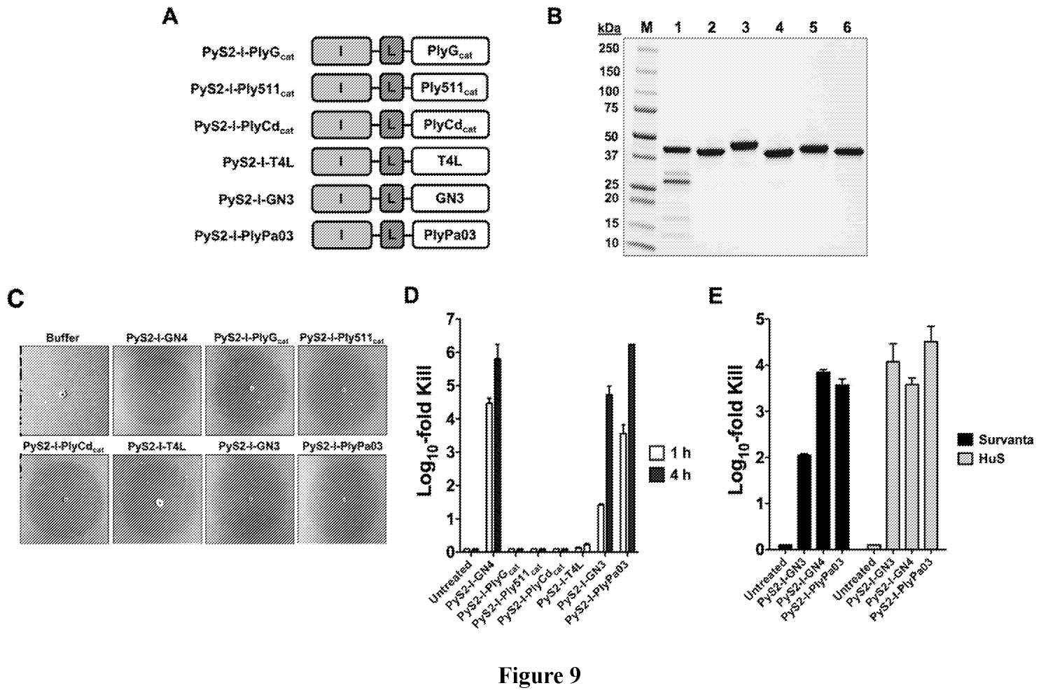

Analyzing the antipseudomonal activity of purified lysocins constructed with different lysins (FIGS. 9A and 9B) revealed that each was capable of exhibiting muralytic activity towards the PG of P. aeruginosa (FIG. 9C). However, domain I of PyS2 was most efficient at delivering Pseudomonas lysins in an enzymatically-active form to the periplasm of target P. aeruginosa (FIG. 9D). The absence of bactericidal activity by lysocins constructed with non-pseudomonal lysins could be explained by either (i) their inability to translocate across the OM, or (ii) failure of the lysin to properly refold into its enzymatically-active form following delivery into the periplasm.

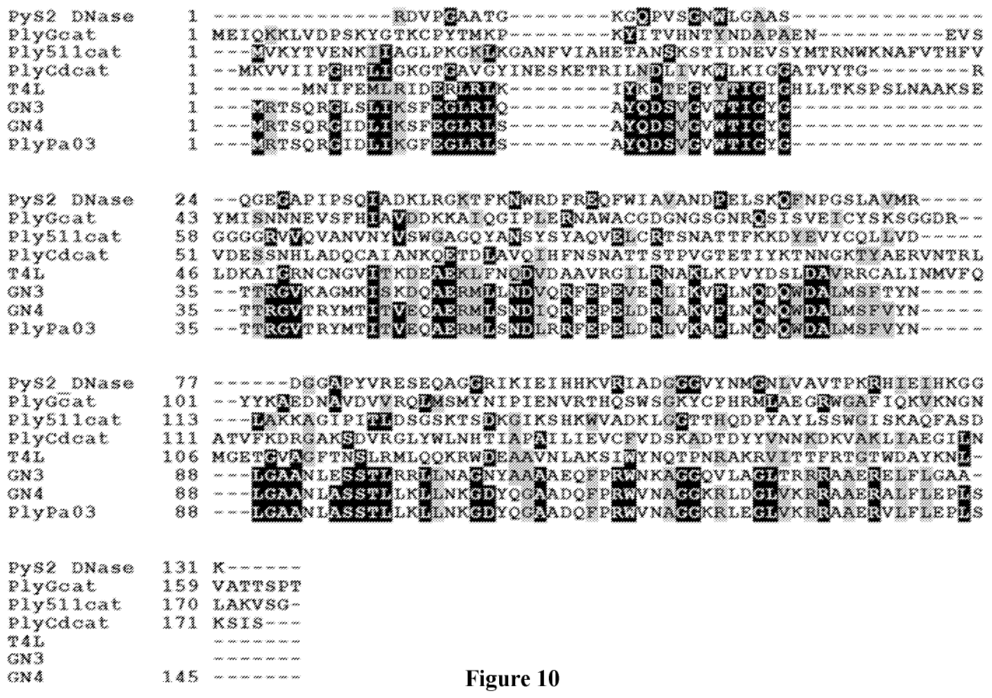

If lysocins comprising non-pseudomonal lysins are incapable of OM translocation, then identifying conserved amino acids and/or biochemical properties between the native PyS2 DNase domain and the Pseudomonas lysins used this disclosure provides information as to why these particular lysins were efficiently delivered across the OM when engineered as a lysocin. Amino acids conserved only between the PyS2 DNase domain and all three of the Pseudomonas lysins were Q23, E62, P72, Q77, G105, R116, G122, G127 and R136 (amino acid coordinates are specific to the three Pseudomonas lysins) (FIG. 10). In a lysocin background, each of the aforementioned conserved amino acids can be individually mutated and then assayed for antipseudomonal activity in order to evaluate the importance of each residue for OM translocation. Comparing general biochemical properties of the PyS2 DNase domain to those of the various lysins used for bioengineering lysocins revealed that only proteins with a molecular weight of ˜16 kDa and less were successfully transported through FpvAI (Table 7). Additional lysins varying in size can be tested in order to determine if there is a firm molecular weight threshold for FpvAI-dependent import. There were no obvious trends specific to the isoelectric point (pI) and grand average of hydropathicity (GRAVY) values to discern lysins that were effective when constructed as lysocins from those that were not. The log₁₀-fold killing of Pseudomonas by PyS2-GN3, PyS2-I-GN4 and PyS2-I-PlyPa03 in the presence of complex matrices, such as growth medium, beractant and serum, highlights the potential therapeutic applicability of lysocins for the treatment of P. aeruginosa skin, lung and bloodstream infections (FIGS. 9D and 9E).

It will be recognized from the figures and description that the present disclosure provides a validated strategy that allows extrinsically-applied lysins to overcome the challenge of both bypassing the OM of P. aeruginosa and exhibiting antibacterial activity in serum. More specifically, the disclosure demonstrates successful bioengineering of a highly specific delivery system that transports functional lysins to their PG substrate in HuS, resulting in PG cleavage and bacterial death. While antibacterial efficacy was confirmed against P. aeruginosa in the presently described non-limiting demonstrations, based on the present disclosure, it is expected that lysocins can be developed to target other antibiotic-resistant Gram-negative bacteria, including E. coli, Y. pestis, and the ESKAPE pathogens K. pneumoniae and E. cloacae; thereby fulfilling an urgent global healthcare need.

TABLE 1
Antimicrobial MIC and MBC values for
numerous P. aeruginosa strains.

| | P. aeruginosa | FpvA | MIC | MBC |
|---|---|---|---|---|
| Antimicrobial | Strain | Type | (μg/ml) | (μg/ml) |
| PyS2-GN4 | PAO1 | I | 2 | — |
| | MDR-M-3 | I | 2 | — |
| | 443 | III | >256 | — |
| | 445 | II | >256 | — |
| | 446 | II | >256 | — |
| | 448 | II | >256 | — |
| | 449 | II | >256 | — |
| | 450 | III | >256 | — |
| | 451 | II | >256 | — |
| | 452 | I | 4 | — |
| | 453 | I | 0.25 | 0.25 |
| Colistin | 453 | I | 0.5 | 0.5 |
| Meropenem | 453 | I | 8 | 8 |
| Piperacillin-Tazobactam | 453 | I | 16 | 128 |
| Tobramycin | 453 | I | 0.125 | 0.25 |

TABLE 2
Antibacterial specificity of PyS2-GN4 against
Gram-positive and Gram-negative bacteria.

| Gram-Positive | | | Gram-Negative | | |
|---|---|---|---|---|---|
| | | MIC | | | MIC |
| Species | Strain | (μg/ml) | Species | Strain | (μg/ml) |
| B. cereus | RSVF1 | >256 | A. baumaneii | ATCC | >256 |
| | | | | 17978 | |
| E. faecium | EFSK-2 | >256 | E. cloacae | NR-50391 | >256 |
| S. aureus | NR- | >256 | E. coli | ATCC | >256 |
| | 45946 | | | 25922 | |
| S. pyogenes | D471 | >256 | K. pneumoniae | NR- | >256 |
| | | | | 41916 | |

TABLE 3
List of bacterial strains used in this study.

| Bacteria | Source |
|---|---|
| A. baumaneii ATCC | ATCC |
| 17978 | |
| B. cereus RSVF1 | Vincent Fischetti, The Rockefeller University |
| E. cloacae NR-50391 | BEI Resources, NIAID, NIH |
| E. coli ATCC 25922 | ATCC |
| E. faecium EFSK-2 | Alexander Tomasz, The Rockefeller University |
| K. pneumoniae NR- | BEI Resources, NIAID, NIH |
| 41916 | |
| P. aeruginosa ATCC | ATCC |
| 15692 | |
| P. aeruginosa 442 | Lars Westblade, Weill Cornell Medical College |
| P. aeruginosa 443 | Lars Westblade, Weill Cornell Medical College |
| P. aeruginosa 445 | Lars Westblade, Weill Cornell Medical College |
| P. aeruginosa 446 | Lars Westblade, Weill Cornell Medical College |
| P. aeruginosa 448 | Lars Westblade, Weill Cornell Medical College |
| P. aeruginosa 449 | Lars Westblade, Weill Cornell Medical College |
| P. aeruginosa 450 | Lars Westblade, Weill Cornell Medical College |
| P. aeruginosa 451 | Lars Westblade, Weill Cornell Medical College |
| P. aeruginosa 452 | Lars Westblade, Weill Cornell Medical College |
| P. aeruginosa 453 | Lars Westblade, Weill Cornell Medical College |
| P. aeruginosa MDR- | Daniel Green, Columbia University Medical |
| M-3 | Center |
| S. aureus NR-45946 | BEI Resources, NIAID, NIH |
| S. pyogenes D471 | The Rockefeller University Lancefield Collection |

TABLE 4
List of primers used in this study

| | | SEQ ID |
|---|---|---|
| Oligonucleotide | Nucleotide Sequence | NO: |
| GN4_F | 5′-AACTTTAAGAAGGAGATATAATGCGCACCAGCCAGCG | 12 |
| | C-3′ | |
| GN4_R | 5′-GTCGACGGAGCTCGAATTCGGATCCTTAGCTCAGCGG | 13 |
| | TTCCAGAAACAGTGC-3′ | |
| PyS2_F | 5′-AACTTTAAGAAGGAGATATACCATGGCCGTGAACGAT | 14 |
| | TATG-3′ | |
| PyS2-GN4_R | 5′-TGGTGCGCATCGGGTCACGAAACATCAC-3′ | 15 |
| PyS2-GN4_F | 5′-TCGTGACCCGATGCGCACCAGCCAGCGC-3′ | 16 |
| PyS2-GN4ΔTBB_F | 5′-[Phos]GTTCAGGGTGGTGGTCGTGACATTATCCAG | 17 |
| PyS2-GN4ΔTBB_R | 5′-[Phos]GCTGCCCGGCTCATAATCGTTCACGGCCATG | 18 |
| PyS2-GN4_KO_1F | 5′-[Phos]CGCCTATCAGGCTAGCGTGGGTGTGTGGACC-3′ | 19 |
| PyS2-GN4_KO_1R | 5′-[Phos]CTCAGGCGCAGGCCCTCAAAGCTCTTAATC-3′ | 20 |
| PyS2-GN4_KO_2F | 5′-[Phos]GCGTGGGTGTGTGGGCCATTGGTTATGGTAC-3′ | 21 |
| PyS2-GN4_KO_2R | 5′-[Phos]TAGCCTGATAGGCGCTCAGGCGCAGGCCC-3′ | 22 |
| FpvAI_F | 5′-CGAAGGCCAGAACTACGAGA-3′ | 23 |
| FpvAI_R | 5′-TGTAGCTGGTGTAGAGGCTCAA-3′ | 24 |
| FpvAII_F | 5′-TACCTCGACGGCCTGCACAT-3′ | 25 |
| FpvAII_R | 5′-GAAGGTGAATGGCTTGCCGTA-3′ | 26 |
| FpvAIII_F | 5′-ACTGGGACAAGATCCAAGAGAC-3′ | 27 |
| FpvAIII_R | 5′-CTGGTAGGACGAAATGCGAG-3′ | 28 |

### Example 9

### Comparing the Antipseudomonal Activity of PyS2-I-GN4 to the Parental Lysocin

Structure- and function-based studies specific to PyS2 indicate that domain I alone (FIG. 1A) is capable of binding FpvAI and translocating across the OM of P. aeruginosa via the TonB import system (51). In the context of PyS2-GN4, this mechanistic understanding suggests domains II and III may not be required for the intracellular delivery of the GN4 lysin and thus could be dispensable for bactericidal activity. To determine if this is accurate, domain I of PyS2 was fused to the GN4 lysin through a short GSx3 linker to generate the truncated lysocin termed PyS2-I-GN4 (FIG. 8A). The antipseudomonal killing kinetics of purified PyS2-I-GN4 were then analyzed in iron-chelated CAA medium and compared to those of the purified full-length parental lysocin PyS2-GN4 (FIGS. 8B and 8C). Untreated and GN4-treated P. aeruginosa were used as negative controls for bactericidal activity. PyS2-I-GN4 was capable of 3.3-log₁₀ killing of P. aeruginosa in 30 min. At 3 h, the lysocin reduced the number of viable bacterial cells to the limit of detection, which is 10 CFU/ml. In contrast, the killing kinetics of PyS2-GN4 were appreciably less than the truncated lysocin. PyS2-GN4 required 3 h to promote a 3.1-log₁₀ decrease in pseudomonal viability and was incapable of lowering the number of viable bacterial cells to the limit of detection over the duration of the experiment. These results indicate that, in addition to maintaining bactericidal activity towards P. aeruginosa, deleting domains II and III from PyS2-GN4 increases the antipseudomonal potency of the lysocin.

### Example 10

### Effect of Iron Availability and Linker Composition on PyS2-I-GN4 Bactericidal Activity

Considering that protein expression level of the FpvA receptor is inversely related to iron availability, the sensitivity of P. aeruginosa to PyS2-I-GN4 was investigated after the bacteria were grown in either iron-rich (CAA medium supplemented with 100 μM FeSO₄) or iron-depleted growth medium (CAA medium containing 0.5 mg/ml EDDHA) (FIG. 8D). Bacteria absent lysocin treatment were used as a negative control for antipseudomonal activity. P. aeruginosa grown using iron-depleted culture conditions were highly sensitive to PyS2-I-GN4 in PBS, with the lysocin generating a 3.6- and 4.7-log₁₀ reduction in pseudomonal viability at 30 min and 6 h, respectively. Following growth using iron-rich culture conditions, P. aeruginosa were largely unsusceptible to PyS2-I-GN4, with only 0.7-log₁₀ killing observed after 6 h. The inverse relationship between iron availability and lysocin sensitivity provides additional evidence that the antimicrobial requires the presence of an iron-regulated OM protein, specifically FpvAI, for OM translocation and subsequent antipseudomonal activity.

In addition to iron availability, the bactericidal properties of PyS2-I-GN4 may be altered by modifying the linker used for fusing domain I of PyS2 to the GN4 lysin. To this end, the bactericidal activity of PyS2-I-GN4, which comprises a GSx3 linker, was compared to that of purified PyS2-I-GN4_{NL} (no linker), PyS2-I-GN4_{12AA} (GSx6 linker) and PyS2-I-GN4_{18AA} (GSx9 linker) (FIGS. 8A, 8B and 8E). Surprisingly, the absence or presence of a linker, as well as its overall length, had no effect on the bactericidal activity of the lysocin. After 2 h, all constructs (PyS2-I-GN4_{NL}, PyS2-I-GN4, PyS2-I-GN4_{12AA} and PyS2-I-GN4_{18AA}) decreased the number of viable P. aeruginosa to the limit of detection in iron-chelated growth medium.

### Example 11

Evaluating Different Lysins for their Potential Use as Lysocins

Similar to PyS2-I-GN4, additional lysocins using domain I of PyS2 can be designed based on the present disclosure to transport other lysins across the OM of P. aeruginosa. As such, the following six lysocins were constructed with a GSx3 linker: PyS2-I-PlyG_{cat}, PyS2-I-Ply511_{cat}, PyS2-I-PlyCd_{cat}, PyS2-I-T4L, PyS2-I-GN3 and PyS2-I-PlyPa03 (FIG. 9A, Table 5). Each purified lysocin (FIG. 9B) was initially spotted on autoclaved Pseudomonas in order to verify that the lysin component was enzymatically active (FIG. 9C). Contrary to the buffer only negative control, all six lysocins degraded pseudomonal PG similar to the PyS2-I-GN4 positive control. However, incubating each lysocin at equal molar concentrations with viable P. aeruginosa revealed that only lysocins constructed with Pseudomonas lysins (i.e., GN3, GN4 (positive control) and PlyPa03) were capable of log₁₀-fold killing at the conclusion of the 4 h experiment (FIG. 9D). At 1 h, PyS2-I-GN3, PyS2-I-GN4 and PyS2-I-PlyPa03 decreased the viability of Pseudomonas 1.4-, 4.5- and 3.6-log₁₀, respectively. All three lysocins were bactericidal after 4 h, with PyS2-I-GN3, PyS2-I-GN4 and PyS2-I-PlyPa03 respectively killing 4.7-, 5.8- and 6.2-log₁₀ P. aeruginosa. These collective results suggest the antipseudomonal killing kinetics of PyS2-I-GN4 and PyS2-I-PlyPa03 are superior to those of PyS2-I-GN3.

Using the broth microdilution assay outlined by CLSI, the antimicrobial susceptibility of 14 different strains of P. aeruginosa to PyS2-I-GN3, PyS2-I-GN4 and PyS2-I-PlyPa03 was determined in iron-chelated CAA medium (Table 6). All seven P. aeruginosa strains that express FpvAI were susceptible to the three lysocins, with corresponding MIC values of ≤8 μg/ml. As expected, due to the requirement of FpvAI for the intracellular delivery of the lysin component, the three lysocins were ineffective towards P. aeruginosa strains expressing FpvAII and FpvAIII

A significant application of lysocins can be expected for use in the treatment of P. aeruginosa lung and/or bloodstream infections. First, lysocin activity against P. aeruginosa was measured in the presence of lung surfactant. Lung surfactant, a prominent component of the alveolar mucosa, is a complex lipid and protein mixture secreted into the alveolar space by epithelial type II cells to minimize the surface tension at the air-liquid interface in the lung (52). In the presence of the pulmonary surfactant beractant (i.e., the composition sold under the trade name SURVANTA), which is a natural bovine lung extract supplemented with artificial surfactants that mimics the composition and surface tension lowering properties of natural lung surfactant, the three lysocins were capable of log₁₀-fold killing of P. aeruginosa after 2 h (FIG. 9E). PyS2-I-GN4 and PyS2-I-PlyPa03 were bactericidal, exhibiting 3.8- and 3.6-log₁₀ killing of P. aeruginosa, whereas PyS2-I-GN3 decreased bacterial viability 2.0-log₁₀. For evaluating antipseudomonal efficacy specific to the treatment of P. aeruginosa BSIs, the bactericidal activity of each lysocin was assayed in HuS. All three lysocins were bactericidal after 2 h, with PyS2-I-GN3, PyS2-I-GN4 and PyS2-I-PlyPa03 displaying 4.1-, 3.6- and 4.5-log₁₀ killing of Pseudomonas, respectively (FIG. 9E).

### Example 12

Bacterial Strains and Growth Conditions Information relating to the P. aeruginosa strains used in this disclosure was previously outlined (67, 68). P. aeruginosa strains 443-453 are clinical isolates from the clinical laboratory of Weill Cornell Medical Center (New York, N.Y.), while strains AR465-AR474 are clinical isolates from New York University Langone Medical Center (New York, N.Y.). Unless stated otherwise, P. aeruginosa were routinely grown in iron-depleted conditions consisting of CAA medium with 0.5 mg/ml EDDHA at 37° C. with aeration for a total of 16-18 h. E. coli were cultured in LB medium at either 18° C. or 37° C. with aeration.

### Molecular Cloning

For pET28a::gn4 and pET28a::pys2-gn4, the PCR conditions and assembly into the E. coli expression vector pET28a were previously described (67). When constructing pET28a::pys2-I-gn4, two independent PCR reactions using the primer pairs PyS2_F/PyS2-I-GN4_R and PyS2-I-GN4_F/GN4_R (Table 8) were initially performed using pET28a::pys2-gn4 as a template. The standard 50 μl PCR mixture consisted of 1 ng template, 1×Q5 Reaction Buffer, 0.2 mM dNTPs, 0.5 μM of each oligonucleotide primer, and 1 U of Q5 DNA polymerase. The standard thermocycler heating conditions consisted of 98° C. for 30 s, 35× (98° C. for 10 s, 60° C. for 30 s, 72° C. for 30 s per kb) and 72° C. for 2 min. The PCR fragments were assembled into pET28a using the NEBuilder HiFi DNA Assembly method. The 20 μl reaction consisting of 1× NEBuilder HiFi DNA Assembly Master Mix, pys2-I-gn4 PCR products, and NcoI/BamHI linearized pET28a was incubated at 50° C. for 15 min and transformed into E. coli DH5a. Following sequence confirmation, pET28a::pys2-I-gn4 was transformed into E. coli BL21(DE3).

PCR coupled with NEBuilder HiFi DNA Assembly was used to generate pET28a::pys2-I-gn4ₙₗ and pET28a::pys2-I-gn4₁₈ₐₐ. For pET28a::pys2-I-gn4ₙₗ, a single PCR reaction utilizing the primer pair PyS2-I-GN4_{NL}_F and PyS2-I-GN4_{NL}_R (Table 8) was used to amplify pET28a::pys2-I-gn4. For pET28a::pys2-I-gn4₁₈ₐₐ, pET28a::pys2-I-gn4 was used as a template for two independent PCR reactions using primer pairs PyS2-I-GN4_{18AA}_Vector_F/PyS2-I-GN4_{18AA}_Vector_R and PyS2-I-GN4_{18AA}_Insert_F/PyS2-I-GN4_{18AA}_Insert_R. The standard PCR reaction and thermocycler conditions were then used.

For DNA assembly, the 20 μl reaction consisting of 1× NEBuilder HiFi DNA Assembly Master Mix and the PCR product(s) was incubated at 50° C. for 15 min and transformed into E. coli DH5a. In order to create pET28a::pys2-I-gn4₁₂ₐₐ, pET28a::pys2-I-gn4 was amplified with the phosphorylated primers PyS2-I-GN4_{12AA}_F and PyS2-I-GN4_{12AA}_R. The 50 μl PCR reaction consisted of 50 ng template, 1×Q5 Reaction Buffer, 0.2 mM dNTPs, 0.5 μM of each oligonucleotide primer, and 1 U of Q5 DNA polymerase. The thermocycler heating conditions consisted of 98° C. for 30 s, 25× (98° C. for 10 s, 60° C. for 30 s, 72° C. for 30 s per kb) and 72° C. for 2 min. The resulting PCR product was ligated using T4 DNA ligase and transformed into E. coli DH5a. Following sequence confirmation, pET28a::pys2-I-gn4ₙₗ, pET28a::pys2-I-gn4₁₂ₐₐ and pET28a::pys2-I-gn4₁₈ₐₐ were transformed into E. coli BL21(DE3).

The templates pET28a::pys2-plyG_{cat}, pET28a::pys2-ply511_{cat}, pET28a::pys2-plyCd_{cat}, pET28a::pys2-t4l, pET28a::pys2-gn3 and pET28a::pys2-plypa03 were previously constructed using the protocol described for generating pET28a::pys2-gn4 (67). Each construct consisted of nucleotides 1-1,674 of pys2 followed by either nucleotides 4-495 of plyG (NC_007734), 4-525 of ply511 (NC_009811, sequence was codon-optimized for expression in E. coli by GeneWiz, Inc.), 4-522 of plyCd (NC_009089), 4-492 of t41 (NC_000866), 4-429 of gn3 (CP000926, sequence was codon-optimized for expression in E. coli by GeneWiz, Inc.) or 4-432 of plypa03. The expression constructs pET28a::pys2-I-plyG_{cat}, pET28a::pys2-I-ply511_{cat}, pET28a::pys2-I-plyCd_{cat}, pET28a::pys2-I-t4l, pET28a::pys2-I-gn3 and pET28a::pys2-I-plypa03 were respectively created using the primers PlyG_{cat}_F and PyS2-I_R (template: pET28a::pys2-plyG_{cat}), Ply511_{cat}_F and PyS2-I_R (template: pET28a::pys2-ply511_{cat}), PlyCd_{cat}_F and PyS2-I_R (template: pET28a::pys2-plyCd_{cat}), T4L_F and PyS2-I_R (template: pET28a::pys2-t4l), GN3_F and PyS2-I_R (template: pET28a::pys2-gn3), and PlyPa03_F and PyS2-I_R (template: pET28a::pys2-plypa03). The 50 μl PCR reactions consisted of 50 ng template, 1×Q5 Reaction Buffer, 0.2 mM dNTPs, 0.5 μM of each oligonucleotide primer, and 1 U of Q5 DNA polymerase. The thermocycler heating conditions consisted of 98° C. for 30 s, 25× (98° C. for 10 s, 60° C. for 30 s, 72° C. for 30 s per kb) and 72° C. for 2 min. The resulting PCR products were ligated using T4 DNA and transformed into E. coli DH5a. Following sequence confirmation, each construct was transformed into E. coli BL21(DE3).

### Protein Expression and Purification

Using E. coli BL21(DE3), all lysocins were expressed at 37° C. for 4 h with shaking at 200 RPM in LB containing 50 μg/ml kanamycin, with one exception. The PyS2-I-PlyCd_{cat} lysocin was expressed at 18° C. for 16 h. Protein expression was induced at mid-log phase (OD_{600 nm}=0.5) with 1 mM IPTG. The cells were then harvested, washed, resuspended in 50 mM Tris-HCl, pH 7.5, consisting of 200 mM NaCl and 1 mM PMSF, and lysed using a Q125 sonicator (Qsonica). The lysate was cleared by centrifugation at 12,000 RPM for 1 h at 4° C. The soluble lysate fraction was dialyzed against 10 mM sodium phosphate, pH 7.0, followed by sterile filtration (0.2 μm) to generate the crude lysate.

The crude lysate was applied to a HiTrap CM FF column in 10 mM sodium phosphate, pH 7.0, using an AKTA FPLC system. Protein was eluted from the column using a linear salt gradient from 0 to 250 mM NaCl. Elution fractions containing the protein of interest were dialyzed against 50 mM Tris-HCl, pH 7.5, 200 mM NaCl, and concentrated using an Amicon Ultracel-10K filter. The protein samples were then applied to a HiLoad 16/60 Superdex 200 Prep Grade column in 50 mM Tris-HCl, pH 7.5, 200 mM NaCl. Following SDS-PAGE analysis, highly pure lysocin elution fractions were combined, concentrated, sterile filtered and stored at −80° C. until further needed.

### Muralytic Assay Using Autoclaved Pseudomonas

P. aeruginosa strain 453 were grown in BHI medium for 16-18 h at 37° C. with aeration. The bacteria were harvested, washed and subsequently diluted 3.2-fold (with respect to the initial culture volume) in 50 mM Tris-HCl, pH 7.5, consisting of 0.75% (wt/vol) agarose. After autoclaving the sample, 10 ml of the bacterial mixture was aliquoted into a 100 mm×15 mm petri dish. For determining muralytic activity, 25 pmol of each purified protein was spotted on the autoclaved P. aeruginosa. Clearing zones observed after an 18 h incubation at 37° C. correspond to muralytic activity. PyS2-I-GN4 was used as a positive control for muralytic activity, while buffer was spotted as a negative control.

### Cell Viability Assays

P. aeruginosa strain 453 were grown under iron-depleted conditions (see Bacterial Strains and Growth Conditions). The bacteria were harvested, washed and resuspended in fresh CAA medium with 0.5 mg/ml EDDHA. Using a 96-well flat-bottomed microtiter plate, the bacteria at ˜10⁶ CFU/ml were incubated statically at 37° C. with either growth medium only (untreated control) or 0.5 μM purified antimicrobial. A 2 h incubation was used for the single time point experiments, whereas a 4 or 6 h incubation was used for the multiple time point assays. At various time points, an aliquot was removed from each sample, serially diluted and plated on CAA agar. After incubating the agar plates for up to 48 h at 37° C., colonies were counted in order to quantitate the CFU/ml concentration of surviving bacterial cells. Modifications were introduced to the aforementioned cell viability assay protocol for the experiment using P. aeruginosa grown in the presence of varying free iron concentrations. First, P. aeruginosa strain 453 were grown using iron-depleted or iron-rich conditions (CAA medium supplemented with 100 μM FeSO₄) at 37° C. with aeration for a total of 16-18 h and subsequently assayed for lysocin sensitivity in PBS, pH 7.4. For the experiment analyzing lysocin activity in the presence of lung surfactant or serum, P. aeruginosa were incubated with or without lysocin in beractant (SURVANTA; Abbvie) or HuS diluted 1:1 with 20 mM sodium phosphate, pH 7.0. Error bars correlate to ±SEM of triplicate experiments.

### MIC Determination

The FpvA receptor type for all P. aeruginosa strains used was determined using multiplex PCR, as previously described (67). The MIC values for PyS2-I-GN3, PyS2-I-GN4 and PyS2-I-PlyPa03 were determined using the CLSI broth microdilution assay (73), with one exception. CAA medium consisting of 0.5 mg/ml EDDHA was used instead of MHB. All P. aeruginosa strains were initially inoculated in CAA medium and grown overnight at 37° C. with aeration. The bacteria were then pelleted, washed and resuspended in CAA medium with EDDHA. Using a 96-well U-bottom microtiter plate, P. aeruginosa at a final concentration of 5×10⁵ CFU/ml were incubated statically at 37° C. with 0.002-256 μg/ml lysocin in CAA medium with EDDHA for a total of 48 h. Growth (bacteria incubated in growth medium absent lysocin) and sterility controls (growth medium only) were included for each dataset. At the culmination of the experiment, each plate was visually inspected for bacterial growth. All samples were assayed in triplicate. The MIC was defined as the lowest concentration of lysocin that inhibited observable bacterial growth.

TABLE 5
Information relating to the amino acid composition of particular lysocins.

| Lysocin | PyS2 AA | Linker | Lysin |
|---|---|---|---|
| PyS2-I-PlyG_{cat} | 1-209 | GSx3 | B. anthracis lysin PlyG catalytic domain (YP_459981, aa 2-165) |
| | | | (70) |
| PyS2-I-Ply511_{cat} | 1-209 | GSx3 | L. monocytogenes lysin Ply511 catalytic domain (ΥP_001468459, |
| | | | aa 2-175) (71) |
| PyS2-I-PlyCd_{cat} | 1-209 | GSx3 | C. difficile lysin PlyCd catalytic domain (YP_001088405, aa 2- |
| | | | 174) (72) |
| PyS2-I-T4L | 1-209 | GSx3 | E. coli lysin T4L (NP_049736, aa 2-164) (73) |
| PyS2-I-GN3 | 1-209 | GSx3 | P. putida lysin GN3 (WP_012273008, aa 2-143) |
| PyS2-I-PlyPa03 | 1-209 | GSx3 | P. aeruginosa lysin PlyPa03 (WP_070344501, aa 2-144) (68) |

The sequence of PlyPa03 is:

(SEQ ID NO: 48)
MRTSQRGIDLIKGFEGLRLSAYQDSVGVWTIGYGTTRGVTRYMTITVEQA
ERMLSNDLRRFEPELDRLVKAPLNQNQWDALMSFVYNLGAANLASSTLLK
LLNKGDYQGAADQFPRWVNAGGKRLEGLVKRRAAERVLFLEPLS

TABLE 6
Antimicrobial susceptibility of 14 different P. aeruginosa strains to PyS2-I-GN3, PyS2-I-GN4 and PyS2-I-PlyPa03 lysocins.

| | MIC (μg/ml) | | | | | | | | | | | | | |
|---|---|---|---|---|---|---|---|---|---|---|---|---|---|---|
| | FpvAI | | | | | | | FpvAII | | | | FpvAIII | | |
| Lysocin | PAO1 | 447 | 453 | AR465 | AR469 | AR470 | AR474 | 445 | 448 | 451 | AR468 | 443 | 450 | AR471 |
| PyS2-I-GN3 | 8 | 8 | 4 | 4 | 8 | 4 | 4 | >256 | >256 | >256 | >256 | >256 | >256 | >256 |
| PyS2-I-GN4 | 4 | 4 | 0.25 | 4 | 8 | 8 | 4 | >256 | >256 | >256 | >256 | >256 | >256 | >256 |
| PyS2-I-PlyPa03 | 4 | 4 | 0.25 | 2 | 4 | 8 | 4 | >256 | >256 | >256 | >256 | >256 | >256 | >256 |

TABLE 7
Biochemical properties of lysin candidates
used for lysocin bioengineering.

| Protein | AA | MW (kDa) | pI | GRAVY |
|---|---|---|---|---|
| PyS2 DNase | 131 | 14.1 | 9.85 | −0.585 |
| PlyG_{cat} | 164 | 18.5 | 8.60 | −0.325 |
| PlyCd_{cat} | 174 | 19.0 | 8.78 | −0.198 |
| Ply511_{cat} | 173 | 19.0 | 9.40 | −0.299 |
| T4L | 163 | 18.6 | 9.59 | −0.399 |
| GN3 | 142 | 15.8 | 9.98 | −0.384 |
| GN4 | 144 | 16.2 | 9.57 | −0.325 |
| PlyPa03 | 143 | 16.1 | 9.74 | −0.333 |

TABLE 8
List of oligonucleotide primers used in this disclosure.

| | | SEQ ID |
|---|---|---|
| Oligonucleotide | Sequence | NO: |
| PyS2_F | 5′-AACTTTAAGAAGGAGATATACCATGGCCGTG | 29 |
| | AACGATTATG-3′ | |
| PyS2-I-GN4_R | 5′-GCTACCGCTGCCGCTACCTTTGTAGTCTGCC | 30 |
| | TCAAC-3′ | |
| PyS2-I-GN4_F | 5′-GGTAGCGGCAGCGGTAGCATGCGCACCAGCC | 31 |
| | AGCGCG-3′ | |
| GN4_R | 5′-GTCGACGGAGCTCGAATTCGGATCCTTAGCT | 32 |
| | CAGCGGTTCCAGAAACAGTGC-3′ | |
| PyS2-I-GN4_{NL}_F | 5′-AGACTACAAAATGCGCACCAGCCAGCGC-3′ | 33 |
| PyS2-I-GN4_{NL}_R | 5′-TGGTGCGCATTTTGTAGTCTGCCTCAACTTC | 34 |
| | TGCTGCG-3′ | |
| PyS2-I-GN4_{12AA}_F | 5′-[Phos]GGTAGCGGCAGCGGTAGCATGCGCA | 35 |
| | CCAGCCAGCGCGGC-3′ | |
| PyS2-I-GN4_{12AA}_R | 5′-[Phos]GCTACCGCTGCCGCTACCTTTGTAG | 36 |
| | TCTGCCTCAACTTC-3′ | |
| PyS2-I-GN4_{18AA}_Vector_F | 5′-GGATCCGAATTCGAGCTC-3′ | 37 |
| PyS2-I-GN4_{18AA}_Vector_R | 5′-GCTACCGCTGCCGCTACCGCTACCGCTG-3′ | 38 |
| PyS2-I-GN4_{18AA}_Insert_F | 5′-GGTAGCGGCAGCGGTAGCGGTAGCGGCAGCG | 39 |
| | GTAGCATGCGCACCAGC-3′ | |
| PyS2-I-GN4_{18AA}_Insert_R | 5′-CGGAGCTCGAATTCGGATCCTTAGCTCAGCG | 40 |
| | GTTCCAGAAAC-3′ | |
| PyS2-IR | 5′-[Phos]GCTACCGCTGCCGCTACCTTTGTAG | 41 |
| | TCTGCCTCAACTTCTG-3′ | |
| PlyG_{cat}_F | 5′-[Phos]GAAATCCAAAAAAAATTAGTTGATC | 42 |
| | CAAGTAAGTATG-3′ | |
| Ply511_{cat}_F | 5′-[Phos]GTGAAATATACCGTGGAAAATAAAA | 43 |
| | TCATCGCCGGCC-3′ | |
| PlyCd_{cat}_F | 5′-[Phos]AAAGTAGTAATAATACCAGGGCACA | 44 |
| | CTTTAATTGG-3′ | |
| T4L_F | 5′-[Phos]AACATCTTCGAAATGCTGCGCATCG | 45 |
| | ACGAACGCCTGCG-3′ | |
| GN3_F | 5′-[Phos]CGCACCAGCCAGCGTGGCCTGAGCC | 46 |
| | TGATTAAGAGC-3′ | |
| PlyPa03_F | 5′-[Phos]CGTACATCCCAACGAGGCATAGACC | 47 |
| | TCATCAAAGGCTTCG-3′ | |

ATCC, American Type Culture Collection;
NIAID, National Institute of Allergy and Infectious Disease;
NIH, National Institutes of Health,
AA, amino acids;
MW, molecular weight;
pI, isoelectric point;
GRAVY, grand average of hydropathicity

### REFERENCES

This reference listing is not an indication that any of the references are material to patentability.
1. McCaughey L C, Josts I, Grinter R, White P, Byron O, Tucker N P, Matthews J M, Kleanthous C, Whitchurch C B, Walker D. 2016. Discovery, characterization and in vivo activity of pyocin SD2, a protein antibiotic from Pseudomonas aeruginosa. Biochem J 473:2345-58.
2. Hattemer A, Hauser A, Diaz M, Scheetz M, Shah N, Allen J P, Porhomayon J, El-Solh A A. 2013. Bacterial and clinical characteristics of health care- and community-acquired bloodstream infections due to Pseudomonas aeruginosa. Antimicrob Agents Chemother 57:3969-75.
3. Lister P D, Wolter D J, Hanson N D. 2009. Antibacterial-resistant Pseudomonas aeruginosa: clinical impact and complex regulation of chromosomally encoded resistance mechanisms. Clin Microbiol Rev 22:582-610.
4. WHO. 2017. Global priority list of antibiotic-resistant bacteria to guide research, discovery, and development of new antibiotics. Organization W H, Geneva. www.who.int/medicines/publications/WHO-PPL-Short_Summary_25Feb-ET_NM_WHO.pdf?ua=1.
5. Fischetti V A. 2010. Bacteriophage endolysins: a novel anti-infective to control Gram-positive pathogens. Int J Med Microbiol 300:357-62.
6. Heselpoth R D, Swift, S. M., Linden, S. B., Mitchell, M. S., Nelson, D. C. 2018. Enzybiotics: Endolysins and Bacteriocins. In al. DHe (ed), Bacteriophages. Springer International Publishing A G.
7 Briers Y, Walmagh M, Van Puyenbroeck V, Cornelissen A, Cenens W, Aertsen A, Oliveira H, Azeredo J, Verween G, Pirnay J P, Miller S, Volckaert G, Lavigne R. 2014. Engineered endolysin-based “Artilysins” to combat multidrug-resistant gram-negative pathogens. MBio 5:e01379-14.
8. Larpin Y, Oechslin F, Moreillon P, Resch G, Entenza J M, Mancini S. 2018. In vitro characterization of PlyE146, a novel phage lysin that targets Gram-negative bacteria. PLoS One 13:e0192507.
9. Thandar M, Lood R, Winer B Y, Deutsch D R, Euler C W, Fischetti V A. 2016. Novel Engineered Peptides of a Phage Lysin as Effective Antimicrobials against Multidrug-Resistant Acinetobacter baumannii. Antimicrob Agents Chemother 60:2671-9.
10. Briers Y, Lavigne R. 2015. Breaking barriers: expansion of the use of endolysins as novel antibacterials against Gram-negative bacteria. Future Microbiol 10:377-90.
11. Yan G, Liu J, Ma Q, Zhu R, Guo Z, Gao C, Wang S, Yu L, Gu J, Hu D, Han W, Du R, Yang J, Lei L. 2017. The N-terminal and central domain of colicin A enables phage lysin to lyse Escherichia coli extracellularly. Antonie Van Leeuwenhoek 110:1627-1635.
12. Lukacik P, Barnard T J, Keller P W, Chaturvedi K S, Seddiki N, Fairman J W, Noinaj N, Kirby T L, Henderson J P, Steven A C, Hinnebusch B J, Buchanan S K. 2012. Structural engineering of a phage lysin that targets gram-negative pathogens. Proc Natl Acad Sci USA 109:9857-62.
13. Chai T, Wu V, Foulds J. 1982. Colicin A receptor: role of two Escherichia coli outer membrane proteins (OmpF protein and btuB gene product) and lipopolysaccharide. J Bacteriol 151:983-8.
14. Cavard D, Lazdunski C. 1981. Involvement of Btub and Ompf Proteins in Binding and Uptake of Colicin-A. Fems Microbiology Letters 12:311-316.
15. Sampson B A, Gotschlich E C. 1992. Elimination of the vitamin B12 uptake or synthesis pathway does not diminish the virulence of Escherichia coli K1 or Salmonella typhimurium in three model systems. Infect Immun 60:3518-22.
16. Hej air HMA, Zhu Y, Ma J, Zhang Y, Pan Z, Zhang W, Yao H. 2017. Functional role of ompF and ompC porins in pathogenesis of avian pathogenic Escherichia coli. Microb Pathog 107:29-37.
17. Ghequire M G, De Mot R. 2014. Ribosomally encoded antibacterial proteins and peptides from Pseudomonas. FEMS Microbiol Rev 38:523-68.
18. Cascales E, Buchanan S K, Duche D, Kleanthous C, Lloubes R, Postle K, Riley M, Slatin S, Cavard D. 2007. Colicin biology. Microbiol Mol Biol Rev 71:158-229.
19. White P, Joshi A, Rassam P, Housden N G, Kaminska R, Goult J D, Redfield C, McCaughey L C, Walker D, Mohammed S, Kleanthous C. 2017. Exploitation of an iron transporter for bacterial protein antibiotic import. Proc Natl Acad Sci USA 114:12051-12056.
20. Joshi A, Grinter R, Josts I, Chen S, Wojdyla J A, Lowe E D, Kaminska R, Sharp C, McCaughey L, Roszak A W, Cogdell R J, Byron O, Walker D, Kleanthous C. 2015. Structures of the Ultra-High-Affinity Protein-Protein Complexes of Pyocins S2 and AP41 and Their Cognate Immunity Proteins from Pseudomonas aeruginosa. J Mol Biol 427:2852-66.
21. Sun Q, Kuty G F, Arockiasamy A, Xu M, Young R, Sacchettini J C. 2009. Regulation of a muralytic enzyme by dynamic membrane topology. Nat Struct Mol Biol 16:1192-4.
22. Kirikae T, Nakano M, Morrison D C. 1997. Antibiotic-induced endotoxin release from bacteria and its clinical significance. Microbiol Immunol 41:285-94.
23. Warren H S, Kania S A, Siber G R. 1985. Binding and neutralization of bacterial lipopolysaccharide by colistin nonapeptide. Antimicrob Agents Chemother 28:107-12.
24. Raymond K N, Dertz E A, Kim S S. 2003. Enterobactin: an archetype for microbial iron transport. Proc Natl Acad Sci USA 100:3584-8.
25. Oliver A, Canton R, Campo P, Baquero F, Blazquez J. 2000. High frequency of hypermutable Pseudomonas aeruginosa in cystic fibrosis lung infection. Science 288:1251-4.
26. Hoffmann N, Rasmussen T B, Jensen P O, Stub C, Hentzer M, Molin S, Ciofu O, Givskov M, Johansen H K, Hoiby N. 2005. Novel mouse model of chronic Pseudomonas aeruginosa lung infection mimicking cystic fibrosis. Infect Immun 73:2504-14.
27. Bone R C. 1991. The pathogenesis of sepsis. Ann Intern Med 115:457-69.
28. Okawa I, Kageyama M, Egami F. 1973. Purification and properties of pyocin S2. J Biochem 73:281-9.
29. Ohkawa I, Shiga S, Kageyama M. 1980. Effect of iron concentration in the growth medium on the sensitivity of Pseudomonas aeruginosa to pyocin S2. J Biochem 87:323-31.
30. Denayer S, Matthijs S, Cornelis P. 2007. Pyocin S2 (Sa) kills Pseudomonas aeruginosa strains via the FpvA type I ferripyoverdine receptor. J Bacteriol 189:7663-8.
31. Smith K, Martin L, Rinaldi A, Raj endran R, Ramage G, Walker D. 2012. Activity of pyocin S2 against Pseudomonas aeruginosa biofilms. Antimicrob Agents Chemother 56:1599-601.
32. Sano Y, Matsui H, Kobayashi M, Kageyama M. 1993. Molecular structures and functions of pyocins S1 and S2 in Pseudomonas aeruginosa. J Bacteriol 175:2907-16.
33. McCaughey L C, Ritchie N D, Douce G R, Evans T J, Walker D. 2016. Efficacy of species-specific protein antibiotics in a murine model of acute Pseudomonas aeruginosa lung infection. Sci Rep 6:30201.
34. Sano Y, Kobayashi M, Kageyama M. 1993. Functional domains of S-type pyocins deduced from chimeric molecules. J Bacteriol 175:6179-85.
35. Bodilis J, Ghysels B, Osayande J, Matthijs S, Pirnay J P, Denayer S, De Vos D,

Cornelis P. 2009. Distribution and evolution of ferripyoverdine receptors in Pseudomonas aeruginosa. Environ Microbiol 11:2123-35.
36. Elfarash A, Dingemans J, Ye L, Hassan A A, Craggs M, Reimmann C, Thomas M S, Cornelis P. 2014. Pore-forming pyocin S5 utilizes the FptA ferripyochelin receptor to kill Pseudomonas aeruginosa. Microbiology 160:261-9.
37. Kageyama M, Kobayashi M, Sano Y, Masaki H. 1996. Construction and characterization of pyocin-colicin chimeric proteins. J Bacteriol 178:103-10.
38. Smith A W, Hirst P H, Hughes K, Gensberg K, Govan J R. 1992. The pyocin Sa receptor of Pseudomonas aeruginosa is associated with ferripyoverdin uptake. J Bacteriol 174:4847-9.
39. Baysse C, Meyer J M, Plesiat P, Geoffroy V, Michel-Briand Y, Cornelis P. 1999. Uptake of pyocin S3 occurs through the outer membrane ferripyoverdine type I I receptor of Pseudomonas aeruginosa. J Bacteriol 181:3849-51.
40. Meyer J M, Neely A, Stintzi A, Georges C, Holder I A. 1996. Pyoverdin is essential for virulence of Pseudomonas aeruginosa. Infect Immun 64:518-23.
41. Takase H, Nitanai H, Hoshino K, Otani T. 2000. Requirement of the Pseudomonas aeruginosa tonB gene for high-affinity iron acquisition and infection. Infect Immun 68:4498-504.
42. Dennis J J, Lafontaine E R, Sokol P A. 1996. Identification and characterization of the tolQRA genes of Pseudomonas aeruginosa. J Bacteriol 178:7059-68.
43. Izano E A, Sadovskaya I, Wang H, Vinogradov E, Ragunath C, Ramasubbu N, Jabbouri S, Perry M B, Kaplan J B. 2008. Poly-N-acetylglucosamine mediates biofilm formation and detergent resistance in Aggregatibacter actinomycetemcomitans. Microb Pathog 44:52-60.
44. de Chial M, Ghysels B, Beatson S A, Geoffroy V, Meyer J M, Pattery T, Baysse C, Chablain P, Parsons Y N, Winstanley C, Cordwell S J, Cornelis P. 2003. Identification of type II and type III pyoverdine receptors from Pseudomonas aeruginosa. Microbiology 149:821-31.
45. Institute CLaS. 2012. Methods for dilution antimicrobial susceptibility tests for bacteria that grow aerobically; approved standard. Clinical and Laboratory Standards Institute, Wayne, Pa.
46. Kalle M, Papareddy P, Kasetty G, Morgelin M, van der Plas M J, Rydengard V, Malmsten M, Albiger B, Schmidtchen A. 2012. Host defense peptides of thrombin modulate inflammation and coagulation in endotoxin-mediated shock and Pseudomonas aeruginosa sepsis. PLoS One 7:e51313.
47. Desmard M, Davidge K S, Bouvet O, Morin D, Roux D, Foresti R, Ricard J D, Denamur E, Poole R K, Montravers P, Motterlini R, Boczkowski J. 2009. A carbon monoxide-releasing molecule (CORM-3) exerts bactericidal activity against Pseudomonas aeruginosa and improves survival in an animal model of bacteraemia. FASEB J 23:1023-31.
48. Felts A G, Giridhar G, Grainger D W, Slunt J B. 1999. Efficacy of locally delivered polyclonal immunoglobulin against Pseudomonas aeruginosa infection in a murine burn wound model. Burns 25:415-23.
49. Shivshetty N, Hosamani R, Ahmed L, Oli A K, Sannauallah S, Sharanbassappa S, Patil S A, Kelmani C R. 2014. Experimental protection of diabetic mice against lethal P. aeruginosa infection by bacteriophage. Biomed Res Int 2014:793242.
50. Dings R P, Haseman J R, Leslie D B, Luong M, Dunn D L, Mayo K H. 2013. Bacterial membrane disrupting dodecapeptide SC4 improves survival of mice challenged with Pseudomonas aeruginosa. Biochim Biophys Acta 1830:3454-7.
51. White P, Joshi A, Rassam P, Housden N G, Kaminska R, Goult J D, Redfield C, McCaughey L C, Walker D, Mohammed S, Kleanthous C. 2017. Exploitation of an iron transporter for bacterial protein antibiotic import. Proc Natl Acad Sci USA 114:12051-12056.
52. Veldhuizen E J, Haagsman H P. 2000. Role of pulmonary surfactant components in surface film formation and dynamics. Biochim Biophys Acta 1467:255-70.
53. Redly G A, Poole K. 2003. Pyoverdine-mediated regulation of FpvA synthesis in Pseudomonas aeruginosa: involvement of a probable extracytoplasmic-function sigma factor, FpvI. J Bacteriol 185:1261-5.
54. Visca P. 2004. Iron regulation and siderophore signalling in virulence by Pseudomonas aeruginosa vol I I. Kluwer Academic Publishers, Dordrecht, Netherlands.
55. Cunliffe H E, Merriman T R, Lamont I L. 1995. Cloning and characterization of pvdS, a gene required for pyoverdine synthesis in Pseudomonas aeruginosa: PvdS is probably an alternative sigma factor. J Bacteriol 177:2744-50.
56. Miyazaki H, Kato H, Nakazawa T, Tsuda M. 1995. A positive regulatory gene, pvdS, for expression of pyoverdin biosynthetic genes in Pseudomonas aeruginosa PAO. Mol Gen Genet 248:17-24.
57. Beare P A, For R J, Martin L W, Lamont I L. 2003. Siderophore-mediated cell signalling in Pseudomonas aeruginosa: divergent pathways regulate virulence factor production and siderophore receptor synthesis. Mol Microbiol 47:195-207.
58. Lamont I L, Beare P A, Ochsner U, Vasil A I, Vasil M L. 2002. Siderophore-mediated signaling regulates virulence factor production in Pseudomonas aeruginosa. Proc Natl Acad Sci USA 99:7072-7.
59. Meyer J M, Neely A, Stintzi A, Georges C, Holder I A. 1996. Pyoverdin is essential for virulence of Pseudomonas aeruginosa. Infect Immun 64:518-23.
60. Spencer R C. 1996. Predominant pathogens found in the European Prevalence of Infection in Intensive Care Study. Eur J Clin Microbiol Infect Dis 15:281-5.
61. Koulenti D, Lisboa T, Brun-Buisson C, Krueger W, Macor A, Sole-Violan J, Diaz E, Topeli A, DeWaele J, Carneiro A, Martin-Loeches I, Armaganidis A, Rello J, Group E-VCS. 2009. Spectrum of practice in the diagnosis of nosocomial pneumonia in patients requiring mechanical ventilation in European intensive care units. Crit Care Med 37:2360-8.
62. Micek S T, Kollef M H, Torres A, Chen C, Rello J, Chastre J, Antonelli M, Welte T, Clair B, Ostermann H, Calbo E, Wunderink R, Menichetti F, Schramm G, Menon V. 2015. Pseudomonas aeruginosa nosocomial pneumonia: impact of pneumonia classification. Infect Control Hosp Epidemiol 36:1190-7.
63. Williams B J, Dehnbostel J, Blackwell T S. 2010. Pseudomonas aeruginosa: host defence in lung diseases. Respirology 15:1037-56.
64. Church D, Elsayed S, Reid O, Winston B, Lindsay R. 2006. Burn wound infections. Clin Microbiol Rev 19:403-34.
65. Coetzee E, Rode H, Kahn D. 2013. Pseudomonas aeruginosa burn wound infection in a dedicated paediatric burns unit. S Afr JSurg 51:50-3.
66. Wisplinghoff H, Bischoff T, Tallent S M, Seifert H, Wenzel R P, Edmond M B. 2004. Nosocomial bloodstream infections in U S hospitals: analysis of 24,179 cases from a prospective nationwide surveillance study. Clin Infect Dis 39:309-17.
67. Heselpoth R D, Euler C W, Schuch R, Fischetti V A. 2019. Lysocins: Bioengineered Antimicrobials That Deliver Lysins across the Outer Membrane of Gram-Negative Bacteria. Antimicrob Agents Chemother 63.
68. Raz A, Serrano A, Hernandez A, Euler C W, Fischetti V A. 2019. Isolation of Phage Lysins That Effectively Kill Pseudomonas aeruginosa in Mouse Models of Lung and Skin Infection. Antimicrob Agents Chemother 63.
69. Institute CLaS. 2012. Methods for dilution antimicrobial susceptibility tests for bacteria that grow aerobically; approved standard. Clinical and Laboratory Standards Institute, Wayne, Pa.
70. Schuch R, Nelson D, Fischetti V A. 2002. A bacteriolytic agent that detects and kills Bacillus anthracis. Nature 418:884-9.
71. Loessner M J, Wendlinger G, Scherer S. 1995. Heterogeneous endolysins in Listeria monocytogenes bacteriophages: a new class of enzymes and evidence for conserved holin genes within the siphoviral lysis cassettes. Mol Microbiol 16:1231-41.
72. Wang Q, Euler C W, Delaune A, Fischetti V A. 2015. Using a Novel Lysin To Help Control Clostridium difficile Infections. Antimicrob Agents Chemother 59:7447-57.
73. Tsugita A, Inouye M. 1968. Purification of bacteriophage T4 lysozyme. J Biol Chem 243:391-7.

The foregoing embodiments and examples are intended to represent non-limiting examples of the disclosure. Routine modifications and equivalents of the embodiments are encompassed.

1. A method for killing Gram-negative bacteria, the method comprising contacting the bacteria with a contiguous polypeptide (a lysocin) that comprises:
i) a bacteriocin segment that can be translocated through an outer membrane channel of the Gram-negative bacteria but is not pesticin or colicin A; and
ii) a lysin catalytic segment that has (PG) hydrolase activity but is not T4L or Lysep3.

2. The method of claim 1, wherein the Gram-negative bacteria are present in a bacterial infection in blood, and/or on skin and/or on lungs and/or the eyes and/or cerebrospinal fluid and/or brain of an individual, and wherein the bacteria are killed.

3. The method of claim 2, wherein the Gram-negative bacterial infection is in the blood of the individual.

4. The method of claim 2, wherein the Gram-negative bacteria infection is in the lungs of the individual.

5. The method of claim 4, wherein the bacterial infection comprises Pseudomonas aeruginosa (P. aeruginosa) as a component of the infection, and wherein the P. aeruginosa are killed.

6. The method of claim 5, wherein the bacteriocin segment comprises an amino acid sequence that is at least 90% identical to the sequence of amino acids 1-209 of SEQ ID NO:10 (Domain I of PyS2), and wherein the bacteriocin segment does not comprise amino acids 559-689 of SEQ ID NO:10 (Domain IV of PyS2).

7. The method of claim 6, wherein the bacteriocin segment further comprises an amino acid sequence that is at least 90% identical to the sequence of amino acids 210-312 of SEQ ID NO:10 (Domain II of PyS2).

8. The method of claim 6, wherein the bacteriocin segment further comprises an amino acid sequence comprising an amino acid sequence that is at least 90% identical to the sequence of amino acids 313-558 of SEQ ID NO:10 (Domain III of PyS2).

9. The method of claim 6, wherein the lysin catalytic segment comprises a segment of a lysin selected from the group of lysins consisting of GN3 lysin, GN4 lysin, PlyG_{cat} lysin, Ply511_{cat} lysin, PlyCd_{cat} lysin, and PlyPa03 lysin.

10. The method of claim 6, wherein the lysin catalytic segment comprises a GN4 lysin segment that comprises an amino acid sequence that is at least 90% identical to SEQ ID NO:11, and wherein optionally, the first Met of SEQ ID NO:11 is omitted.

11. The method of claim 10, wherein the GN4 lysin segment comprises the amino acid sequence of SEQ ID NO:11, and wherein optionally, the first Met of SEQ ID NO:11 is omitted.

12. The method of claim 1, wherein the PyS2 comprises at least the sequence of amino acids 1-209 of SEQ ID NO:10 and wherein the GN4 lysin segment comprises the sequence of SEQ ID NO:11, wherein optionally, the first Met of SEQ ID NO:11 is omitted.

13. A contiguous polypeptide comprising:
i) a bacteriocin segment that can be translocated through an outer membrane channel of the Gram-negative bacteria but is not pesticin or colicin A; and
ii) a lysin catalytic segment that has peptidoglycan (PG) hydrolase activity but is not T4L or Lysep3.

14. The contiguous polypeptide of claim 13, wherein bacteriocin segment comprises a segment of P. aeruginosa bacteriocin pyocin S2 (PyS2).

15. The contiguous polypeptide of claim 14, wherein the PyS2 comprises an amino acid sequence that is at least 90% identical to the sequence of 1-209 of SEQ ID NO:10 (Domain I of PyS2), and wherein the bacteriocin segment does not comprise amino acids 559-689 of SEQ ID NO:10 (Domain IV of PyS2).

16. The contiguous polypeptide of 15, wherein the bacteriocin segment further comprises an amino acid sequence that is at least 90% identical to the sequence of amino acids 210-312 of SEQ ID NO:10 (Domain II of PyS2).

17. contiguous polypeptide of claim 15, wherein the bacteriocin segment further comprises an amino acid sequence comprising an amino acid sequence that is at least 90% identical to the sequence of amino acids 313-558 of SEQ ID NO:10 (Domain III of PyS2).

18. The contiguous polypeptide of claim 15, wherein the lysin catalytic segment comprises a segment of any lysin that has PG hydrolase activity, particularly lysins from the group of lysins comprising GN3 lysin, GN4 lysin, PlyG_{cat} lysin, Ply511_{cat} lysin, PlyCd_{cat} lysin, and PlyPa03 lysin.

19. The contiguous polypeptide of claim 18, wherein the lysin catalytic segment is from the GN4 lysin, and wherein the lysin catalytic segment comprises an amino acid sequence that is at least 90% identical to SEQ ID NO:11, and wherein optionally, the first Met of SEQ ID NO:11 is omitted.

20. The contiguous polypeptide of claim 19, wherein the PyS2 comprises an amino acid sequence that is at least 90% identical to the sequence of amino acids 1-209 of SEQ ID NO:10, and wherein the GN4 lysin segment comprises an amino acid sequence that is at least 90% identical to SEQ ID NO:11, and wherein optionally, the first Met of SEQ ID NO:11 is omitted.

21. The contiguous polypeptide of claim 18, wherein the PyS2 comprises at least amino acids 1-209 of SEQ ID NO:10, and wherein the GN4 lysin segment comprises SEQ ID NO:11, and wherein optionally, the first Met of SEQ ID NO:11 is omitted.

22. An expression vector encoding a contiguous polypeptide of claim 13.

23. A method comprising expressing the expression vector of claim 22 in a cell culture, and optionally separating at least one contiguous polypeptide encoded by the expression vector from the cell culture.

24. A pharmaceutical formulation comprising a contiguous polypeptide of claim 13.

25. The pharmaceutical formulation of claim 24, wherein the pharmaceutical formulation comprises a mucoadhesive agent, or is comprised by a spray, a gel, or a cream for topical delivery.


SEQUENCE LISTING (48 sequences)

SEQ ID NO: 1 - protein, 6 aa, Artificial Sequence
  linker sequence
GSGSGS

SEQ ID NO: 2 - protein, 131 aa, Pseudomonas aeruginosa
RDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 3 - protein, 165 aa, Bacillus anthracis
MEIQKKLVDPSKYGTKCPYTMKPKYITVHNTYNDAPAENEVSYMISNNNEVSFHIAVDDKKAIQGIPLERNAWACGDGNGSGNRQSISVEICYSKSGGDRYYKAEDNAVDVVRQLMSMYNIPIENVRTHQSWSGKYCPHRMLAEGRWGAFIQKVKNGNVATTSPT

SEQ ID NO: 4 - protein, 175 aa, Listeria monocytogenes
MVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSG

SEQ ID NO: 5 - protein, 174 aa, Clostridium difficile
MKVVIIPGHTLIGKGTGAVGYINESKETRILNDLIVKWLKIGGATVYTGRVDESSNHLADQCAIANKQETDLAVQIHFNSNATTSTPVGTETIYKTNNGKTYAERVNTRLATVFKDRGAKSDVRGLYWLNHTIAPAILIEVCFVDSKADTDYYVNNKDKVAKLIAEGILNKSIS

SEQ ID NO: 6 - protein, 164 aa, Escherichia coli
MNIFEMLRIDERLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSIWYNQTPNRAKRVITTFRTGTWDAYKNL

SEQ ID NO: 7 - protein, 143 aa, Pseudomonas putida
MRTSQRGLSLIKSFEGLRLQAYQDSVGVWTIGYGTTRGVKAGMKISKDQAERMLLNDVQRFEPEVERLIKVPLNQDQWDALMSFTYNLGAANLESSTLRRLLNAGNYAAAAEQFPRWNKAGGQVLAGLTRRRAAERELFLGAA

SEQ ID NO: 8 - protein, 144 aa, Pseudomonas aeruginosa PAJU2
MRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 9 - protein, 144 aa, Pseudomonas aeruginosa
MRTSQRGIDLIKGFEGLRLSAYQDSVGVWTIGYGTTRGVTRYMTITVEQAERMLSNDLRRFEPELDRLVKAPLNQNQWDALMSFVYNLGAANLASSTLLKLLNKGDYQGAADQFPRWVNAGGKRLEGLVKRRAAERVLFLEPLS

SEQ ID NO: 10 - protein, 689 aa, Pseudomonas aeruginosa
MAVNDYEPGSMVITHVQGGGRDIIQYIPARSSYGTPPFVPPGPSPYVGTGMQEYRKLRSTLDKSHSELKKNLKNETLKEVDELKSEAGLPGKAVSANDIRDEKSIVDALMDAKAKSLKAIEDRPANLYTASDFPQKSESMYQSQLLASRKFYGEFLDRHMSELAKAYSADIYKAQIAILKQTSQELENKARSLEAEAQRAAAEVEADYKARKANVEKKVQSELDQAGNALPQLTNPTPEQWLERATQLVTQAIANKKKLQTANNALIAKAPNALEKQKATYNADLLVDEIASLQARLDKLNAETARRKEIARQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 11 - protein, 144 aa, Pseudomonas aeruginosa PAJU2; identical to SEQ ID NO: 8

SEQ ID NO: 12 - DNA, 38 bp, Artificial Sequence
  primer
aactttaagaaggagatataatgcgcaccagccagcgc

SEQ ID NO: 13 - DNA, 52 bp, Artificial Sequence
  primer
gtcgacggagctcgaattcggatccttagctcagcggttccagaaacagtgc

SEQ ID NO: 14 - DNA, 41 bp, Artificial Sequence
  primer
aactttaagaaggagatataccatggccgtgaacgattatg

SEQ ID NO: 15 - DNA, 28 bp, Artificial Sequence
  primer
tggtgcgcatcgggtcacgaaacatcac

SEQ ID NO: 16 - DNA, 28 bp, Artificial Sequence
  primer
tcgtgacccgatgcgcaccagccagcgc

SEQ ID NO: 17 - DNA, 30 bp, Artificial Sequence
  primer
gttcagggtggtggtcgtgacattatccag

SEQ ID NO: 18 - DNA, 31 bp, Artificial Sequence
  primer
gctgcccggctcataatcgttcacggccatg

SEQ ID NO: 19 - DNA, 31 bp, Artificial Sequence
  primer
cgcctatcaggctagcgtgggtgtgtggacc

SEQ ID NO: 20 - DNA, 30 bp, Artificial Sequence
  primer
ctcaggcgcaggccctcaaagctcttaatc

SEQ ID NO: 21 - DNA, 31 bp, Artificial Sequence
  primer
gcgtgggtgtgtgggccattggttatggtac

SEQ ID NO: 22 - DNA, 29 bp, Artificial Sequence
  primer
tagcctgataggcgctcaggcgcaggccc

SEQ ID NO: 23 - DNA, 20 bp, Artificial Sequence
  primer
cgaaggccagaactacgaga

SEQ ID NO: 24 - DNA, 22 bp, Artificial Sequence
  primer
tgtagctggtgtagaggctcaa

SEQ ID NO: 25 - DNA, 20 bp, Artificial Sequence
  primer
tacctcgacggcctgcacat

SEQ ID NO: 26 - DNA, 21 bp, Artificial Sequence
  primer
gaaggtgaatggcttgccgta

SEQ ID NO: 27 - DNA, 22 bp, Artificial Sequence
  primer
actgggacaagatccaagagac

SEQ ID NO: 28 - DNA, 20 bp, Artificial Sequence
  primer
ctggtaggacgaaatgcgag

SEQ ID NO: 29 - DNA, 41 bp, Artificial Sequence; identical to SEQ ID NO: 14
  primer

SEQ ID NO: 30 - DNA, 36 bp, Artificial Sequence
  primer
gctaccgctgccgctacctttgtagtctgcctcaac

SEQ ID NO: 31 - DNA, 37 bp, Artificial Sequence
  primer
ggtagcggcagcggtagcatgcgcaccagccagcgcg

SEQ ID NO: 32 - DNA, 52 bp, Artificial Sequence; identical to SEQ ID NO: 13
  primer

SEQ ID NO: 33 - DNA, 28 bp, Artificial Sequence
  primer
agactacaaaatgcgcaccagccagcgc

SEQ ID NO: 34 - DNA, 38 bp, Artificial Sequence
  primer
tggtgcgcattttgtagtctgcctcaacttctgctgcg

SEQ ID NO: 35 - DNA, 39 bp, Artificial Sequence
  primer
ggtagcggcagcggtagcatgcgcaccagccagcgcggc

SEQ ID NO: 36 - DNA, 39 bp, Artificial Sequence
  primer
gctaccgctgccgctacctttgtagtctgcctcaacttc

SEQ ID NO: 37 - DNA, 18 bp, Artificial Sequence
  primer
ggatccgaattcgagctc

SEQ ID NO: 38 - DNA, 28 bp, Artificial Sequence
  primer
gctaccgctgccgctaccgctaccgctg

SEQ ID NO: 39 - DNA, 48 bp, Artificial Sequence
  primer
ggtagcggcagcggtagcggtagcggcagcggtagcatgcgcaccagc

SEQ ID NO: 40 - DNA, 42 bp, Artificial Sequence
  primer
cggagctcgaattcggatccttagctcagcggttccagaaac

SEQ ID NO: 41 - DNA, 41 bp, Artificial Sequence
  primer
gctaccgctgccgctacctttgtagtctgcctcaacttctg

SEQ ID NO: 42 - DNA, 37 bp, Artificial Sequence
  primer
gaaatccaaaaaaaattagttgatccaagtaagtatg

SEQ ID NO: 43 - DNA, 37 bp, Artificial Sequence
  primer
gtgaaatataccgtggaaaataaaatcatcgccggcc

SEQ ID NO: 44 - DNA, 35 bp, Artificial Sequence
  primer
aaagtagtaataataccagggcacactttaattgg

SEQ ID NO: 45 - DNA, 38 bp, Artificial Sequence
  primer
aacatcttcgaaatgctgcgcatcgacgaacgcctgcg

SEQ ID NO: 46 - DNA, 36 bp, Artificial Sequence
  primer
cgcaccagccagcgtggcctgagcctgattaagagc

SEQ ID NO: 47 - DNA, 40 bp, Artificial Sequence
  primer
cgtacatcccaacgaggcatagacctcatcaaaggcttcg

SEQ ID NO: 48 - protein, 144 aa, Pseudomonas aeruginosa; identical to SEQ ID NO: 9